(19) United States
(12) Patent Application Publication
(10) Pub. No.: US 2015/0320954 A1
(43) Pub. Date: Nov. 12, 2015

(54) CPAP DEVICE

(71) Applicant: NIDEC COPAL ELECTRONICS CORPORATION, Tokyo (JP)

(72) Inventors: Takashi SUZUKI, Iruma-shi (JP); Takashi KANAI, Iruma-shi (JP); Yuki NAKADA, Iruma-shi (JP); Kiyoshi ARIFUKU, Iruma-shi (JP); Yasuhiro TOBINAI, Iruma-shi (JP); Takayuki ENDO, Iruma-shi (JP); Takatoshi INOGUCHI, Iruma-shi (JP); Masatoshi OBAYASHI, Iruma-shi (JP); Naoya EGUCHI, Iruma-shi (JP)

(73) Assignee: NIDEC COPAL ELECTRONICS CORPORATION, Tokyo (JP)

(21) Appl. No.: 14/652,269

(22) PCT Filed: Sep. 30, 2013
(86) PCT No.: PCT/JP2013/005828

§ 371 Date: Jun. 15, 2015

(30) Foreign Application Priority Data
Dec. 17, 2012 (JP) 2012-274449
Aug. 9, 2013 (JP) 2013-166391

(51) Int. Cl. A61M 16/00; A61M 16/08; A61M 16/06

(52) U.S. Cl. CPC A61M 16/0066; A61M 16/0003; A61M 16/0666; A61M 16/0683; A61M 16/0816; A61M 2016/0027; A61M 2016/003; A61M 2205/3334; A61M 2205/3546; A61M 2205/42

(57) ABSTRACT

The present invention relates to a CPAP apparatus, and obtains compatibility between convenience of handling and easing of loads to a patient is obtained in a high order. An air blowing unit 10 including a fan provided with an air dynamic bearing, and a hose connecting the air blowing unit with a nasal cannula or a mask 200 are included, and the air blowing unit 10 is supported at a position away from a head 310 of a patient 300 by other than the hose 20, and the hose 20 follows changing of posture of the patient 300, and the air blowing unit 10 changes a position or a posture thereof by receiving a force via the hose 20.

### TECHNICAL FIELD

The present invention is related to a CPAP (Continuous Positive Airway Pressure) apparatus which is used for treatment of Sleep Apnea Syndrome.

### BACKGROUND ART

For treatment of Sleep Apnea Syndrome, there have been used CPAP apparatuses which forcibly send air into the respiratory tract by a fan while putting a nasal cannula or a mask to a face. As such a CPAP apparatus, there has been generally adopted a configuration in which a main unit which includes a fan, a control section and the like is placed at a position away from a human body, and between the main unit and the mask or the like which is put on a face is connected by a hose of about 1.5 meters and air is sent in through the hose. Nasal cannulas or masks which have various shapes or are formed by various materials have been developed and put onto the market, and a patient arbitrarily chooses and uses a mask which fits for its face shape and matches its preferences.

In a case of a CPAP apparatus of such configuration, there are a number of problems such as one in which the apparatus requires a hose having a length as long as 1.5 meters, and its main unit has a volume of the order of 140 mm×180 mm×100 mm, since such apparatus is inconvenient for a patient to handle it, contrary to that the treatment method is required to be used every day, such apparatus becomes one of treatment apparatuses which are often not used.

In Patent Literature 1, there are proposed some configurations in order to make the CPAP apparatus be easy in handling.

In other words, in the Patent Literature 1, as one example, there is illustrated a configuration in which a fan and a mask are placed integrally in front of a face.

However, in the case of this configuration, an apparatus which has considerably a lot of volume and considerably a lot of weight is put on a face. In addition, in this configuration, since vibrations as the fan rotates are directly transmitted to the face, and in addition, sound noise of the rotation of the fan are heard immediately near an ear and so on, there is a possibility in which sleeping is rather disturbed.

In addition, in this Patent Literature 1, there is also illustrated a configuration in which the apparatus including the fan is put on away from the body of a patient, specifically, on a belt worn on a west or an arm of the patient, between the apparatus and the mask put on the face is connected by a hose. In this case, a hose having a length fairly shorter than a hose having a length as long as 1.5 meters which is used in the conventional CPAP apparatuses may be used. In addition, since the apparatus is away from a face, loads on a patient seem to be smaller than those of the configuration in which the apparatus and the hose are integrally put on a face. However, when the apparatus is fixed to a patient body, there are possibilities in which vibrations as the fan rotates which fan is provided in the apparatus are directly transmitted to the body of the patient. It is conceivable that a measure of preventing vibrations is applied so that the vibrations are not transmitted to a body of a patient, and however, there may be produced other problems such as one that the volume is increased for that portion and the apparatus becomes inconvenient for handling.

In addition, in a CPAP apparatus, a fan is rotated according to breathing of a patient, air flows as the fan rotates, and sound noise are produced as the fan rotates and the air flows. A CPAP apparatus is an apparatus which is used while a patient is sleeping, it is especially required to be silent, and how to reduce the noise becomes a problem.

As a proposal to aim reducing noise with respect to a CPAP apparatus, for example, in Patent Literature 2, there is disclosed that a chamber to reduce noise is provided.

However, in this case, the camber itself becomes large in size, and the problem of downsizing is not solved.

In addition, in Patent Literature 3, there is disclosed a configuration in which an inlet-side silencer and an outlet-side silencer are arranged at an inlet side and an outlet side of an air blowing device, respectively.

However, in this Patent Literature 3, there is not shown a specific structure and specific material of the inlet-side silencer and the outlet-side silencer, and in addition, it seems to be a proposal without considering anything about reducing the size as a whole including the air blowing device.

Incidentally, in the present invention, which will be described later, a fan which includes an air dynamic bearing which is one form of the fluid dynamic bearing is used, and there are presented here the literatures (the Patent Literatures 4 and 5) in which the air dynamic bearing is disclosed.

### PRIOR ART LITERATURES

Patent Literature 1: Japanese Laid-open Patent Publication No. 2011-156410

Patent Literature 2: Japanese Laid-open Patent Publication No. H7-275362

Patent Literature 3: Japanese National Publication of International Patent Application No. 2002-537006

Patent Literature 4: Japanese Laid-open Patent Publication No. 2007-57048

Patent Literature 5: Japanese Laid-open Patent Publication No. 2009-52485

### ABSTRACT OF THE INVENTION

### Technical Problem

In view of the foregoing, it is an object of the invention to provide a CPAP apparatus in which compatibility between convenience of handling and easing of loads to a patient is obtained in a high order.

### Solution to Problem

A CPAP apparatus according to the present invention to achieve the above-described object includes:

an air blowing unit that includes a housing which has an air suction port, and that includes a fan which has an air receiving port and an air blowing port, is provided with a fluid dynamic bearing, and receives air from the receiving port which air is suctioned from the suction port into the housing to send out the air from the air blowing port; and

a hose that connects the air blowing unit with an air intake port of a nasal cannula or a mask which is attached to the head of a patient so as to cover an external naris or a nose of the patient and supplies the air taken in from the air intake port to a respiratory tract of the patient, and that sends the air sent out from the air blowing unit from the air intake port to the nasal cannula or the mask, wherein

the air blowing unit is supported at a position away from the head of the patient by other than the hose, and the hose is a hose having such a length that exerts a force to the air blowing unit via the hose when the patient changes a posture thereof while lying, and the air blowing unit receives the force to change a position or a posture thereof.

In the CPAP apparatus according to the present invention, the air blowing unit is provided with the fan having the fluid dynamic bearing. For this reason, the air blowing unit is significantly downsized and reduced in weight.

As such, the CPAP apparatus according to the present invention is made to has a configuration in which the air blowing unit is placed immediately near a patient such as a bedside of the patient, and between a nasal cannula or a mask is connected by a short hose, and when the patient changes its posture such as turning over, a force is exerted to the air blowing unit through the hose, and the air blowing unit also follows the posture changes to change a position or a posture thereof.

According to the CPAP apparatus according to the present invention, since the hose is shortened compared to a conventional one, the handling is easy, and since when a patient does turning over and the like in its bed the air blowing unit changes its position or posture while following the turning over and the like, loads to the patient while using the CPAP apparatus are reduced.

Here, in the CPAP apparatus according to the present invention, it is preferable that the air blowing unit further includes a control circuit which receives an instruction by wireless communication and controls the fan based on the instruction, and

the CPAP apparatus further comprises a remote controller that gives an instruction to the control circuit by wireless communication.

For a patient to give instructions to the control circuit, for example, instructions of a selection between a fixed mode and an automatic mode, a target air pressure and the like, an operation button and the like may be provided in the air blowing unit, and however, when the remote controller is provided, it is possible to perform operations without reaching a hand thereof to the air blowing unit, and thus loads on a patient are reduced in terms of operability.

In addition, in the CPAP apparatus according to the present invention, it is also a preferable configuration that the CPAP apparatus further includes a movable joint between the air blowing unit and the hose.

Although the air blowing unit is reduced in weight and in size by applying the fan of the fluid dynamic bearing, there exist its weight and volume thereof. And so, by connecting the air blowing unit to the hose via the movable joint, some posture changes of a patient may be absorbed by moving of the movable joint and the air blowing unit itself may not be requited to move, and loads on a patient who is a user are further reduced.

Further, in the CPAP apparatus according to the present invention, it is preferable that the housing includes a plurality of air suction ports, and that the housing includes a guard section which prevents the air suction port from being blocked.

In the CPAP apparatus according to the present invention, it is supposed that the air blowing unit changes its position or posture when the CPAP apparatus is being used. However, when the air blowing unit changes its position or the posture and the air suction port may be blocked, there are possibilities in which functions as a CPAP apparatus may be lowered. By providing the plural air suction ports or arranging a guard section to prevent the air suction port form being blocked, the tolerance to a change of the position or posture of the air blowing unit is improved.

In addition, in the CPAP apparatus according to the present invention, it is preferable that the air blowing unit further includes a discharge silencer which reduces noise as the air flows which air is sent out from the air blowing port by the fan coupled to the air blowing port flows.

As described above, the fan provided with the fluid dynamic bearing is used in the CPAP apparatus according to the present invention. This fan may be rotated significantly faster compared to a fan which is conventionally applied to a CPAP apparatus. For this reason, a diameter of a blade required to obtain a required pressure and air flow volume is greatly reduced, and the weight is also significantly reduced. In a CPAP apparatus of conventional type, as one example, a fan which includes a blade having a diameter of 53 mm and has weight of approximately 240 g, and if a fan of the fluid dynamic bearing is applied, for example, a fan which has a blade having a diameter of 29 mm and has weight of approximately 40 g may merely be required.

However, in a case in which a fan including a fluid dynamic bearing is applied, the fan is required to rotate faster compared to a conventional fan, specifically, at the time of inspiration, it is required to further increase the rotation speed in order to increase the air flow volume, and thus noises become large. It is observed that these noises are transmitted from a blowing side of the fan through a flow path to a patient.

In addition, since an amount of changing of the rotation speed of the fan is also increased as the air flow amount change by breathing of a patient, changing of the noises by the increase of the rotation speed of the fan (changing of frequencies of the noises and changing of noise levels) also increases, and thus resulting in more harsh noises.

The present invention aims downsizing and reducing in weight by applying the fan of fluid dynamic bearing, and when the discharge silencer is further provided at a side of blowing out air, the CPAP apparatus in which compatibility between downsizing, reducing in weight and reducing noise is achieved in a high order is obtained.

Here, in the CPAP apparatus according to the present invention, it is preferable that the discharge silencer includes a sound absorbing member made of foamed material.

By forming the discharge silencer with the sound absorbing member made of foamed material, the discharge silencer is also reduced in size and saved in weight, and thus the CPAP apparatus is further reduced in size and saved in weight as a whole.

Further, in a case in which the sound absorbing member made of foamed material is used, since there are effects of reducing noises of a broad frequency band, compared to the chamber configuration described in the Patent Literature 2, it is effective specifically to noises including broad frequency components such as wind noise.

In addition, in the CPAP apparatus according to the present invention, it is preferable that the air blowing unit further includes a suction silencer which includes a sound absorbing member in which a suction path to guide the air suctioned into the housing from the air suction port to the air receiving port, and supports the fan such that the suction silencer contains the fan.

When the sound absorbing member is included, and the suction silencer to support the fan such that the suction silencer enfolds the fan is provided, the CPAP apparatus becomes an apparatus in which both of the noises as air is suctioned and vibrations of the fan are reduced.

Further, in the CPAP apparatus according to the present invention, it is preferable that the air blowing port and the discharge silencer are connected with each other by a joint formed by an elastic body.

When between the air blowing port of the fan and the discharge silencer is connected by the joint formed by the elastic body, vibration transmission of the fan to the discharge silencer is reduced, and noises are further reduced.

### Advantageous Effects of Invention

As explained above, according to the CPAP apparatus according to the present invention, compatibility between convenience of handling and easing of loads to a patient is obtained in a high order.

### BRIEF DESCRIPTION OF THE DRAWINGS

FIG. 1 is a perspective view of a whole configuration of a CPAP apparatus as a first embodiment.

FIG. 2 is an explanatory diagram illustrating a usage condition of the CPAP apparatus illustrated in FIG. 1.

FIG. 3 is an exploded perspective view of the CPAP apparatus according to the first embodiment whose external view is illustrated in FIG. 1.

FIG. 4 is a transparent view of the CPAP apparatus according to the first embodiment when viewed obliquely from above.

FIG. 5 is a transparent view of the CPAP apparatus according to the first embodiment when viewed from side.

FIG. 6 is a control block diagram of the CPAP apparatus according to the embodiment.

FIG. 7 is a perspective view of a turbofan.

FIG. 8 is a plan view of the turbofan.

FIG. 9 is an exploded perspective view of the turbofan viewed obliquely from above.

FIG. 10 is an exploded perspective view of the turbofan viewed obliquely from below.

FIG. 11 is a view illustrating a blade which is a part of the turbofan.

FIG. 12 is a sectional view of the turbofan in a direction indicated by arrows A-A in FIG. 8.

FIG. 13 is a view illustrating a usage condition of a CPAP apparatus according to a second embodiment.

FIG. 14 is a control block diagram of the CPAP apparatus according to the second embodiment illustrated in FIG. 13.

FIG. 15 is a view illustrating a first alternative example of the second embodiment.

FIG. 16 is a view illustrating a second alternative example of the second embodiment.

FIG. 17 is an exploded perspective view of an air suction port of the second alternative example illustrated in FIG. 16.

FIG. 18 is a view illustrating a third alternative example of the second embodiment.

FIG. 19 is a sectional perspective view of an air blowing unit of the third alternative example illustrated in FIG. 18.

FIG. 20 is a perspective view of a CPAP apparatus according to a third embodiment.

FIG. 21 is a transparent perspective view illustrating an air blowing unit of the CPAP apparatus according to the third embodiment illustrated in FIG. 20.

FIG. 22 is a sectional view of the air blowing unit of the CPAP apparatus according to the third embodiment.

FIG. 23 is a view illustrating a usage condition of a CPAP apparatus according to a fourth embodiment.

FIG. 24 is a view illustrating a usage condition of a CPAP apparatus according to a fifth embodiment.

FIG. 25 is a view illustrating a usage condition of a CPAP apparatus according to a sixth embodiment.

FIG. 26 is a view illustrating a usage condition of a CPAP apparatus according to a seventh embodiment.

FIG. 27 is an exploded perspective view illustrating a CPAP apparatus according to a eighth embodiment.

FIG. 28 is a transparent view when the CPAP apparatus according to the eighth embodiment is viewed obliquely from above.

FIG. 29 is a sectional view along arrows A-A illustrated in FIG. 28 of the CPAP apparatus according to the eighth embodiment.

FIG. 30 is a transparent view when the case and the suction silencer are removed from the CPAP apparatus according to the eighth embodiment, and a fan, a discharge structure body and the like are viewed obliquely from above.

FIG. 31 is a control block diagram of the CPAP apparatus according to the eighth embodiment.

FIG. 32 is a schematic diagram of an experimental equipment.

FIG. 33 is a view illustrating noises of fans of a comparative example and an embodiment when the pressure is 1.2 kPa and the flow amount is 50 L/min (litter/minute).

FIG. 34 is a view illustrating noises of fans of the comparative example and the embodiment when the pressure is 1.2 kPa and the flow amount is 110 L/min.

FIG. 35 is a view illustrating noises of the fan of the comparative example at the time when breathing stops and at the time of inspiration.

FIG. 36 is a view illustrating noises of the fan of the embodiment at the time when breathing stops and at the time of inspiration.

FIG. 37 is a view illustrating differences between noise levels of the fan of the embodiment and noise levels of the fan of the comparative example when breathing stops.

FIG. 38 is a view illustrating differences between noise levels of the fan of the embodiment and noise levels of the fan of the comparative example at the time of inspiration.

FIG. 39 is a view illustrating changes of noise levels when a length of the discharge silencer is changed at the time of inspiration.

FIG. 40 is a view illustrating noise levels at 7 kHz with respect to the length of the sound absorbing member included in the discharge silencer which noise levels are read and obtained from the FIG. 39.

FIG. 41 is a view illustrating changes of noise levels when the thickness of the discharge silencer is changed when breathing.

FIG. 42 is a view illustrating noise levels at 1 kHz which noise levels are read from the FIG. 41.

FIG. 43 is a view illustrating noise levels at 3.5 kHz which noise levels are read from the FIG. 41.

FIG. 44 is a view illustrating noise levels at 5.5 kHz which noise levels are read from FIG. 41.

FIG. 45 is a transparent view when the case and the suction silencer are removed from the CPAP apparatus according to the ninth embodiment, and a fan, a discharge silencer and the like are viewed obliquely from above.

FIG. 46 is an exploded perspective view of a CPAP apparatus according to a tenth embodiment.

FIG. 47 is a sectional view of an air blowing unit of the CPAP apparatus whose exploded perspective view is illustrated in FIG. 46.

FIG. 48 is a sectional view of a fan and a discharge silencer of a CPAP apparatus according to a eleventh embodiment.

FIG. 49 is a sectional view of a fan and a discharge silencer of a CPAP apparatus according to a twelfth embodiment.

### BEST MODE FOR CARRYING OUT THE INVENTION

In the following, embodiments of the present invention will be described.

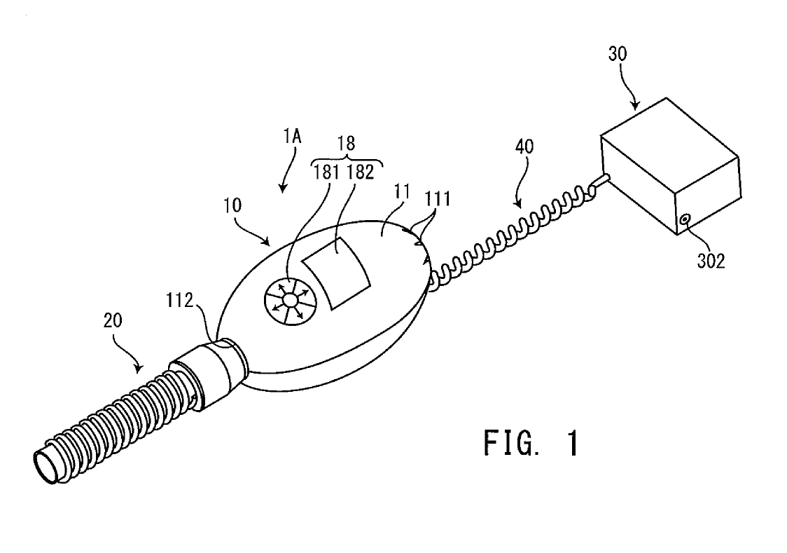

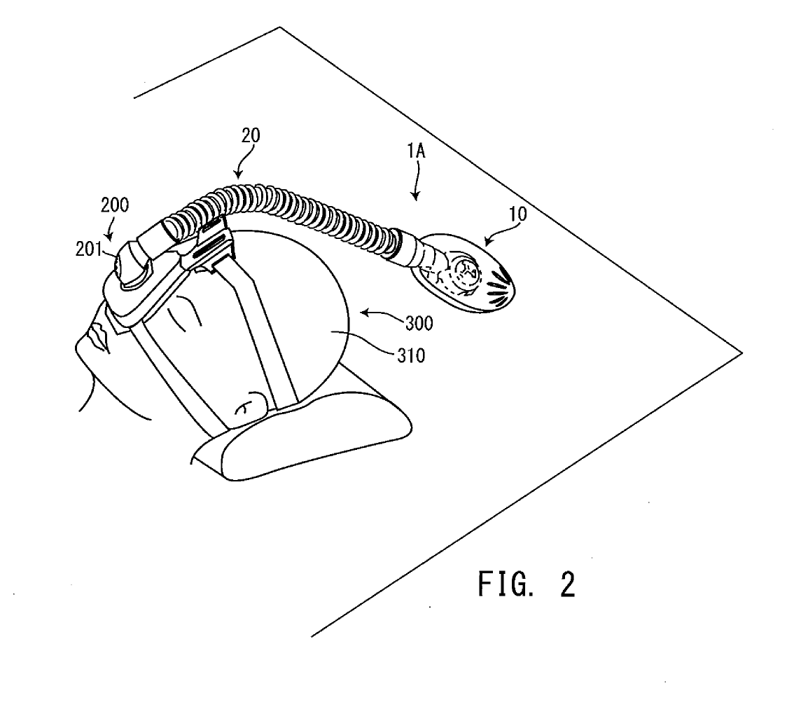

FIG. 1 is a perspective view of a whole configuration of a CPAP apparatus as a first embodiment, and FIG. 2 is an explanatory view illustrating a usage condition of the CPAP apparatus illustrated in FIG. 1. However, in FIG. 2, illustration of a battery case 30 and a cable 40 which are illustrated in FIG, 1 is omitted. In addition, in this FIG. 2, with respect to an air blowing unit, a transparent view indicating a schematic of an inside thereof is illustrated.

This CPAP apparatus 1A includes an air blowing unit 10, a hose 20, a battery case 30 and a cable 40. This CPAP apparatus 1A is used, as illustrated in FIG. 2, in a condition in which the air blowing unit 10 and a mask 200 are connected by the hose 20, the mask 200 is attached to a face of a patient 300 and the air blowing unit 10 is placed at a position away from a head 310 of the patient 300, in here, in a state in which the air blowing unit 10 is placed at a patient's bed side. Accordingly, the hose 20 has a hose whose length is, for example, the order of 50 cm. Plural air suction ports 111 are provided in a case 11 as a housing in which the air blowing unit is housed, and in addition, a fan which will be described later is provided in the case 11. When the fan rotates, air is sent into the mask 200 via the hose 20. The air sent into the mask 200 is supplied to a respiratory tract of the patient 300. Breath is emitted to the outside from a leaking apertures 201 provided in the mask 200. The air blowing unit 10 according to the present embodiment has an oval spherical shape as a whole, and when the patient 300 wearing the mask 200 changes its posture while keeping its lying posture, for example, when the patient turns over on its bed, a force when the posture is changed is transmitted to the air blowing unit 10 via the hose 20, the air blowing unit 10 rolls or slides and thus a position or a posture of the air blowing unit 10 is also changed according to the posture of the patient.

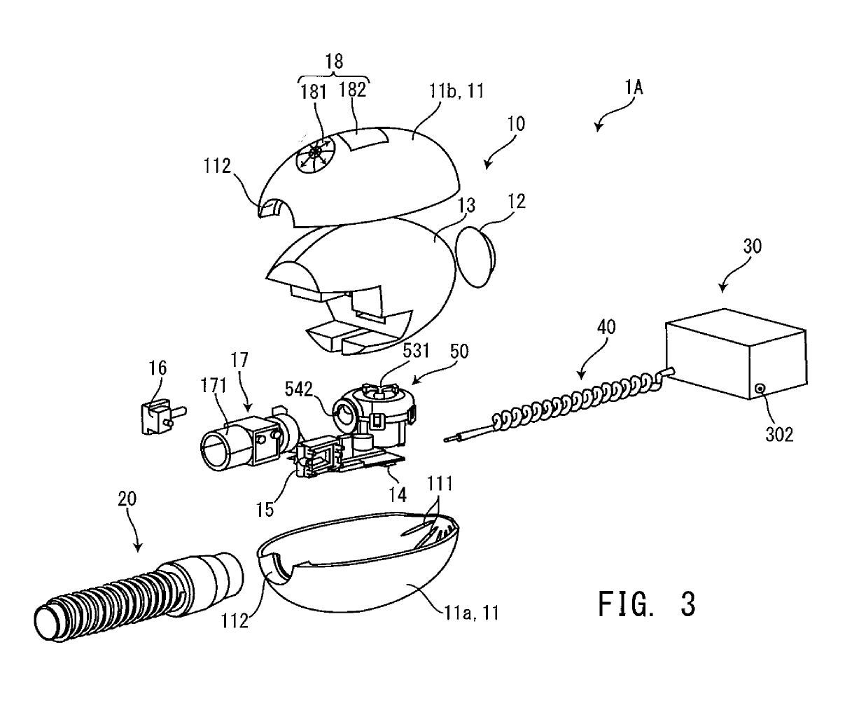

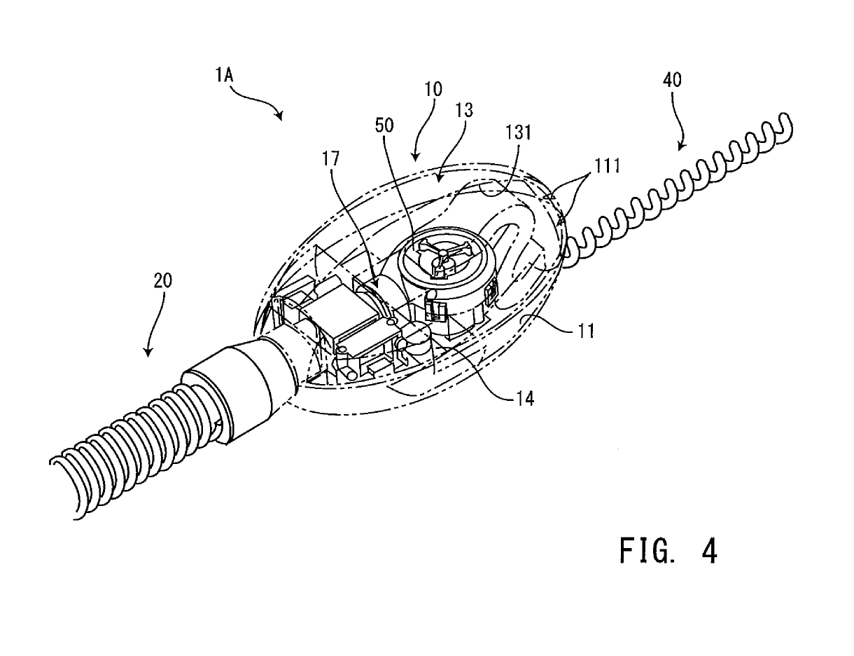

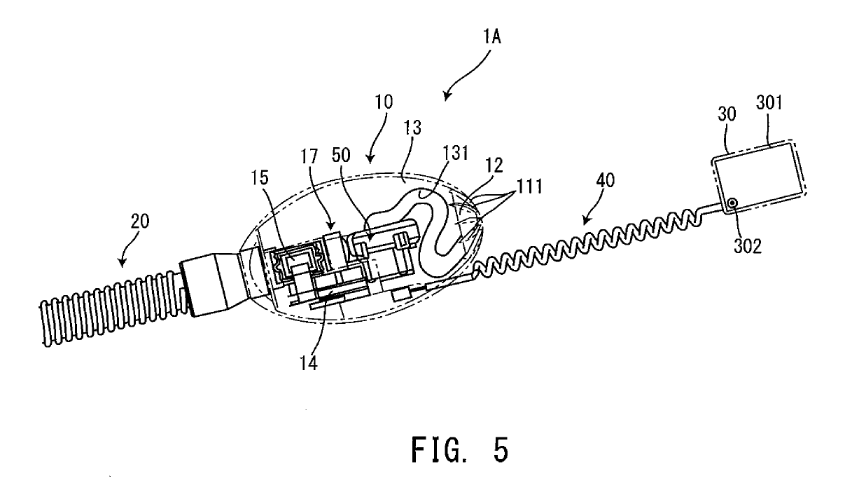

FIG. 3 is an exploded perspective view of the CPAP apparatus according to the first embodiment whose external view is illustrated in FIG. 1. In addition, FIG. 4 is a transparent view of the CPAP apparatus according to the first embodiment when viewed obliquely from above, and FIG. 5 is a transparent view of the CPAP apparatus according to the first embodiment when viewed from side.

In this CPAP apparatus 1A according to the first embodiment, the case 11 of the air blowing unit 10 is configured with a case lower section 11a and a case upper section 11b which are illustrated in FIG. 1.

Since the case 11 has the oval spherical shape as a whole, the case easily rolls. In addition, this case 11 is made of plastic and its external surface is formed to be smooth, and the case easily moves slidably. In order that the air suction is not disturbed even if this case 11 rolls or slides, this case 11 is provided with the plural air suction ports 111.

In addition, the case upper section 11a is provided with a user interface 18 including an operation button 181 and a display screen 182.

An air filter 12, a suction silencer 13, a control board 14, a flow sensor 15, a pressure sensor 16, a discharge path 17 and a turbofan 50 as the fan are arranged in the case 11.

In addition, this CPAP apparatus 1A includes, as described above, the hose 20, the battery case 30 and the cable 40.

The air filter 12 is arranged immediately inside the air suction ports 111 provided in the case 11, and is a filter which absorbs dusts in the air suctioned from the air suction ports 111.

In addition, the air suction silencer 13 has a air flow path 131 which turns as illustrated in FIG. 4 and FIG. 5, and plays a role as a noise reduction mechanism to reduce suctioning noise of the air which is suctioned from the air suction ports 111.

The turbofan 50 receives the air which is suctioned form the air suction ports 111 of the case 11 and comes via the air filter 12 and the silencer 13, and sends out the air from the air blowing port 542.

The control board 14 calculates a rotation setting speed of the turbofan 50 according to an initial setting by a doctor or a patient, a flow amount measured by the flow sensor and a pressure measured by the pressure sensor 16, and gives an instruction to the turbofan 50 to rotate at the rotation speed.

The flow sensor 15 and the pressure sensor 16 are sensors which measure a flow amount and a pressure of the air sent out from the turbofan 50, respectively.

Further, the discharge path 17 is an air path to connect the air blowing port 542 of the turbofan 50 and the air discharge port 112 of the case 11, and an end section 171 on a side of the air discharge port 112 is a part which plays a role of connecting to the hose 20.

A battery 301 is housed in the battery case 30, as illustrated in FIG. 5, electric power from the battery 301 is supplied to the air blowing unit 10 via the cable 40. A battery terminal 302 to which an AC adapter (not shown) for charging the inside battery 301 is provided in this battery case 30. The battery 301 is a component having a considerable volume and a considerable weight, and in order to make the air blowing unit 10 compact and lightweight, in here, a configuration in which the battery case 30 which is separate from the air blowing unit 10 is provided and is connected by the cable 40 is applied. However, without the battery case 30 and the large battery 301 are not provided, and a configuration in which an AC adapter is connected to the air blowing unit 10 to cause the air blowing unit 10 to operate may be applied.

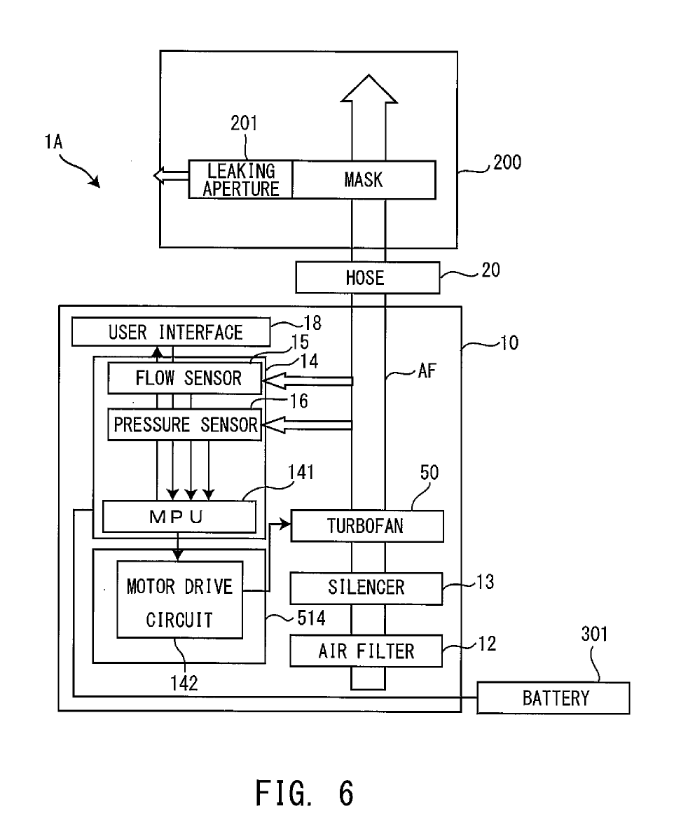

FIG. 6 is a control block diagram of the CPAP apparatus according to the first embodiment.

In here, an air flow path AF which passes from the air blowing unit 10 via the hose 20 through the mask 200 and a control system of the air blowing unit 10 are illustrated.

As described above, the air filter 12, the silencer 13 and the turbofan 50 are arranged on the air flow path AF in the air blowing unit 10, and when the turbofan 50 rotates, air is suctioned from the air suction ports 111 (see, for example, FIG. 5), dusts in the air are removed by the air filter 12, noises as the air is suctioned are reduced by the silencer 13, the air is sent into the mask 200 via the hose 20 by the rotation of the turbofan 50. The air sent into the mask 200 is sent to a respiratory tract of a patient by the inspiration of the patient, and is discharged through the leaking apertures 201 to the outside by the expiration of the patient.

The air blowing unit 10 is provided with a user interface 18 including an operation button 181 and a display screen 182 (see, for example, FIG. 1). The patient operates the operation button 181 while checking the display screen 182, and sets a selection between a fixed mode and an automatic mode, a pressure range of air sent out from the turbofan 50 which pressure range is designated by a doctor, on-off timing of the turbofan 50 and the like. Here, the fixed mode is a mode in which a pressure of air sent out from the turbofan 50 is fixed to a designated pressure, and the automatic mode is a mode in which a state of breathing of a patient is detected by changes of flow amounts or pressures by the flow sensor 15 or the pressure sensor 16, the pressure is changed in the designated range according to the state of breathing of the patient.

Information set by the user interface 18 is input into an MPU (Micro Processing Unit) 141. In addition, air flow amounts and air pressures measured by the flow sensor 15 and the pressure sensor 16 are also input into the MPU 141. The MPU 141 calculates a rotation speed of the turbofan 50 based on the those pieces of the information. A result of the calculation by the MPU 141 is sent to the motor drive circuit 142, and the motor drive circuit 142 drives the turbofan 50 based on the result of the calculation.

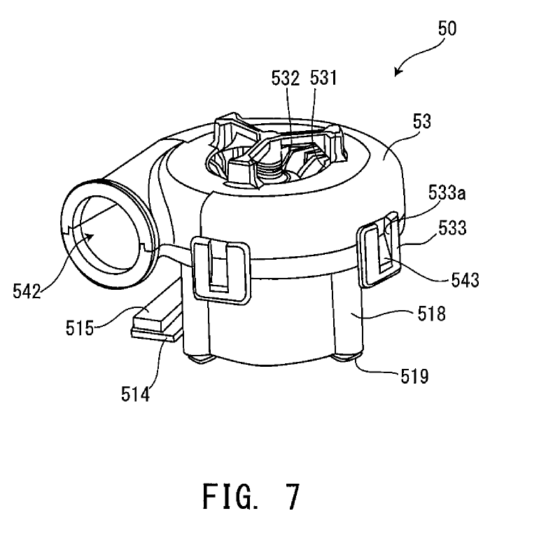

The flow sensor 15, the pressure sensor 16 and the MPU 141 are mounted on the control board 14 housed in the air blowing unit 10. Electrical power is supplied to the control board 14 from the battery 301, the electrical power is distributed to each of sections which require the electrical power. In addition, the motor drive circuit 142 is mounted on the circuit board 514 (see, for example, FIG. 7) which is integrally provided with the turbofan 50.

One of characteristics of the present embodiment is in that the turbofan 50 provided with the air dynamic bearing is applied. Thanks to this, the CPAP apparatus 1A according to the present embodiment 1A succeeds in making the air blowing unit 10 downsized and lightweight significantly.

Here, the turbofan provided with the air dynamic bearing which is applied to the CPAP apparatus 1A according to the present embodiment will be explained. The turbofan which is explained here is same in terms of the operation principals as those disclosed in the above-described Patent Literatures 4 and 5.

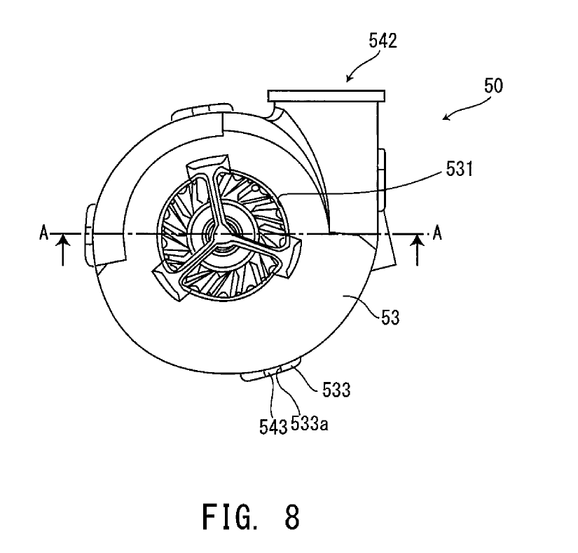

FIG. 7 is a perspective view of a turbofan, and FIG. 8 is a plan view of the turbofan.

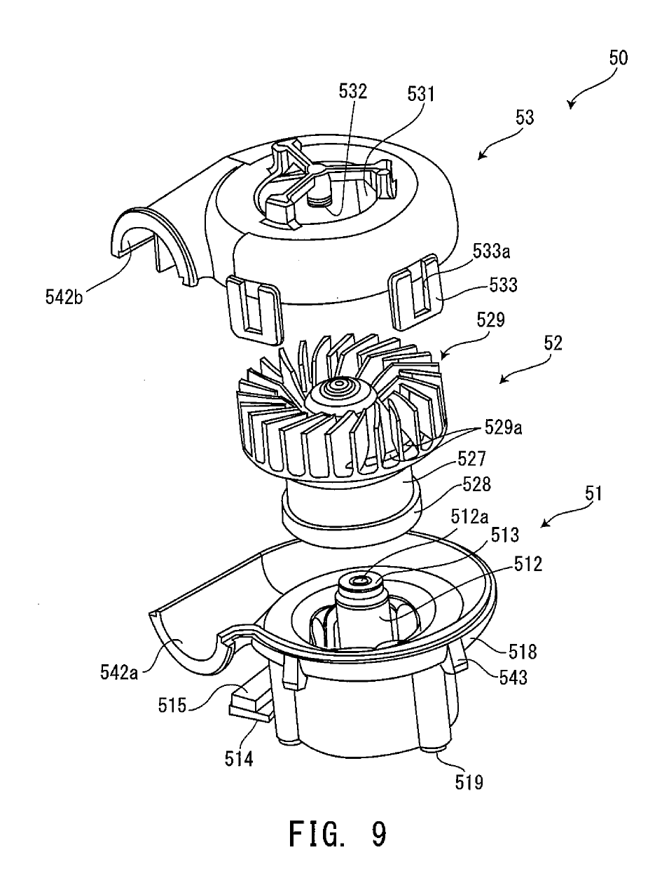

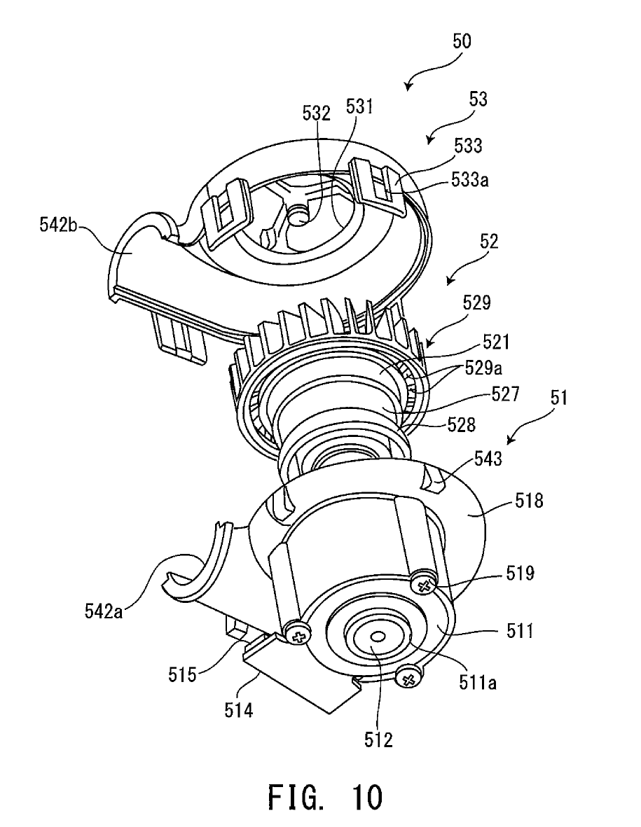

In addition, FIG. 9 and FIG. 10 are an exploded perspective view of the turbofan viewed obliquely from above and obliquely from below, respectively.

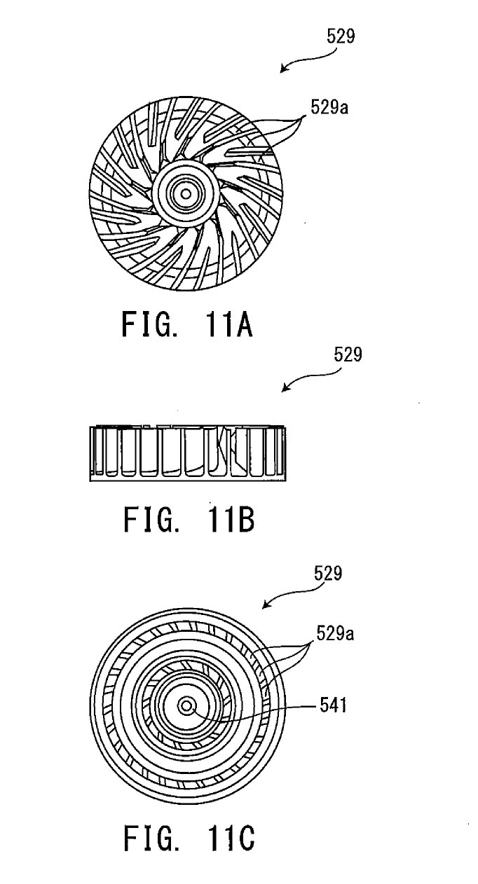

Further, FIG. 11 is a view illustrating a blade 529 which is a part of the turbofan 50. Part (A), part (B) and part (C) of FIG. 11 are a plan view, a side view and a bottom view, respectively.

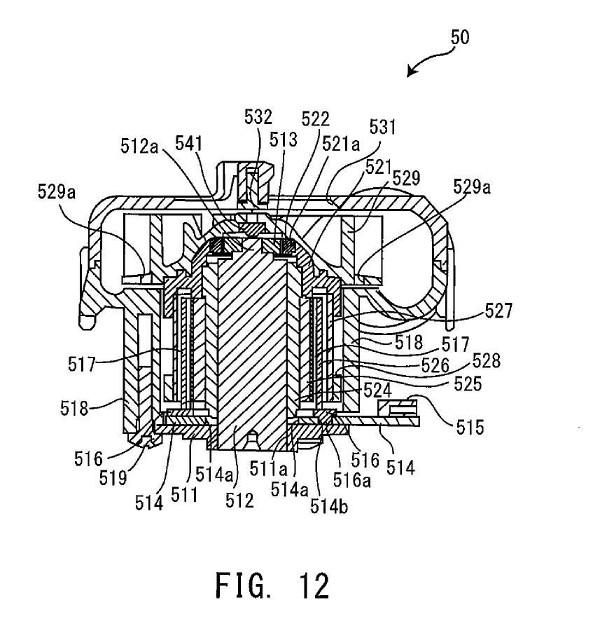

Furthermore, FIG. 12 is a sectional view of the turbofan 50 in a direction indicated by arrows A-A in FIG. 8.

In here, a configuration of this turbofan 50 will be explained while mainly referring to the sectional view of FIG. 12, and referring to other drawings as required.

As illustrated in FIG. 9 and FIG. 10, when roughly divided, this turbofan 50 includes, a stator 51, a rotor 52 and an upper cover 53.

The stator 51 includes a shaft base 511 having ring shape as a base, and a lower portion of a shaft 512 fits into an hole 511a in a center of the shaft base 511 having the ring shape to be fixed. An upper end portion 512a of this shaft 512 is formed to have a small diameter, and a thrust magnet (inside) 513 having a ring shape is fixed such that the upper portion 512a fits thereto. In addition, the circuit board 514 is placed on the shaft base 511. This circuit board 514 is formed with an hole 514a to allow the shaft 512 to go therethrough, and spreads to surround the shaft 512. In addition, this circuit board 514 spreads such that a portion thereof is extended off to the outside, and a connector 515 for connecting to an external circuit is arranged on the extended-off portion.

In addition, a coil base 516 having a ring shape which coil base surrounds the shaft 512 while being slightly away from the shaft 512 is placed on this circuit board 514. In the coil base 516, leg sections 516a which go into holes 514b provided in the circuit board 514 and are supported by the shaft base 511 are arranged at plural positions in a circumferential direction. In other words, this coil base 516 has a shape as a whole in which the coil base 516 is supported at the leg sections 516a by the shaft base 511, and circles on an upper surface of the circuit board 514 around the shaft 512 as a center.

Further, a coil 517 which is formed to have a cylindrical shape as a whole is put on this coil base 516, and a lower end of the coil 517 is fixed to the coil base 516. Electrical power of three-phase pulse is supplied to this coil 517.

In addition, a case 518 is screwed by screws 519 to this shaft base 511.

The rotor 52 has a hub 521 as a base. An hole 521a is formed in an upper portion of this hub 521, a thrust magnet (outside) 522 having a ring shape is fixed to an edge of the hole 521a. An internal circumferential surface of this thrust magnet (outside) 522 faces an external circumferential surface of the thrust magnet (inside) 513 across a significantly small gap therebetween, and a contact between a sintered body 541 and the shaft upper portion 512a in a thrust direction is avoided by an absorbing force between their magnetic forces.

In addition, a sleeve 524 having a cylindrical shape is fixed to this hub 521. An internal circumferential surface of this sleeve 524 faces an external circumferential surface of the shaft 512, a significantly small gap in order of μm is formed between the sleeve 524 and the shaft 512.

A magnet 525 is fixed to an external circumferential surface of this sleeve 524, and a reinforcing ring 526 is attached to an external circumferential surface of the magnet 525. Since the rotor 52 of the turbofan 50 rotates in a high speed, there is a possibility in which the magnet 525 is cracked by a centrifugal force, and the reinforcing ring 526 is for preventing such crack. An external circumferential surface of this reinforcing ring 526 faces an internal circumferential surface of the coil 517 across a narrow space therebetween. Further, on a side of an external circumferential surface of the coil 517, a back yoke 527 is arranged with a space between the coil 517 and the back yoke 527. This back yoke 527 forms a magnetic circuit together with the magnet 525 to play a role of increasing an interaction with the coil 517. A balance ring 528 is fixed to a bottom portion of this back yoke 527. This balance ring 528 is a member for adjusting a balance when the rotor 52 rotates.

In addition, a blade 529 (see also FIG. 11 together) is fixed to an upper portion of the hub 521. The blade 529 is a component which sends out air by the rotation of the rotor 52.

Further, a sintered body 541 is fixed to a lower central section of the blade 529. The sintered body 541 is for causing an air gap between the stator 51 and rotor 2 to have a damper effect, and since when the rotor 52 is going to move in the thrust direction it is possible to prevent an abrupt movement of the rotor 52 by this damper effect, it makes it possible that the rotor 52 may rotate in a high speed in a non-contact manner with respect to the stator 51. In addition, the sintered body 541 is placed at a position facing the upper end section 512a of the shaft 512 of the stator 51. This plays a role of preventing the blade 529 and the like from being damaged while allowing the sintered body 541 to abut against an upper surface of the shaft 512, when, with respect to the sintered body 541, for example, an air resistance at the air blowing side is increased and pressure difference between the top and bottom of the blade 529 is produced, and the blade 529 moves to a side of the rotor 51 by the pressure difference. In addition, a bypass opening 529a is formed in the blade 529. When an air resistance on the side of air blowing rises or a side of air taking is blocked, air flows through the bypass opening 529a, and thus, the bypass opening 529a plays a role of reducing a pressure difference between the inside and the outside of the blade 529, thereby preventing movements of the blade and the like.

As illustrated in FIG. 9 and FIG. 10, an air receiving port 531 is provided in an upper section of the upper cover 53, and, in a side section thereof, there is formed a half cylinder section 542b which forms an air blowing port 542 having a cylindrical shape together with a half cylinder section 542a on a side of the stator 51.

Locking holes 533a provided in locking sections 533 which are formed to protrude downwardly on a side surface and a locking projection 543 formed on a side surface of the upper cover 53 and locking projections 543 which are formed on a side surface of the case 518 of the stator 51 are engaged with each other, so that this upper cover 53 is fixed to the case 518 of the stator 51 in a state in which a small space is formed with respect to the blade 529. A stopper 532 which is exposed downwardly is provided in a center of this upper cover 53. When, for example, a state is produced in which the air receiving hole 531 is blocked or a more upstream side is blocked so that air does not flow into the air receiving port 531 is produced, the rotor 52 tends to float by a pressure difference between the inside and the outside of the blade 529, and at this time, the stopper 53 is for preventing the blade 529 from being damaged by allowing the upper central portion of the blade 529 to abut against this stopper 532.

This turbofan 50 includes the above-described configuration, electric power of three-phase pulses is applied to the coil 517, and the rotor 52 rotates according to a cycling frequency of the three-phase pulses.

Here, this turbofan 50 has the configuration in which there is no contact between the stator 51 and the rotor 52 and the air dynamic bearing is arranged between them, and it is suitable for high-speed rotation, is small in diameter and lightweight, and may produce an air flow amount required as a CPAP apparatus.

Incidentally, the turbofan 50 provided with an air dynamic bearing is explained here, and however, a fan which may be applied in the CPAP apparatus according to the present invention is not necessarily provided with an air dynamic bearing, and in general, a fan as far as provided with a fluid dynamic bearing, such as one in which between a stator and a rotor is filled with oil, may be used.

This ends the explanations of the CPAP apparatus 1A according to the first embodiment, and in the following, a second embodiment and embodiments after the second embodiment will be explained. Incidentally, in the drawings illustrating each of the second embodiment and the embodiments after the second embodiment, for the convenience of understanding, components and the like functionally corresponding to those included in the CPAP apparatus 1A according to the first embodiment even though there are differences in shapes and the like are illustrated while provided with reference signs same as those assigned in each of the drawings used for the explanations of the first embodiment which is described above, and the explanations will be concentrated to configuration portions distinctive to each of the embodiments.

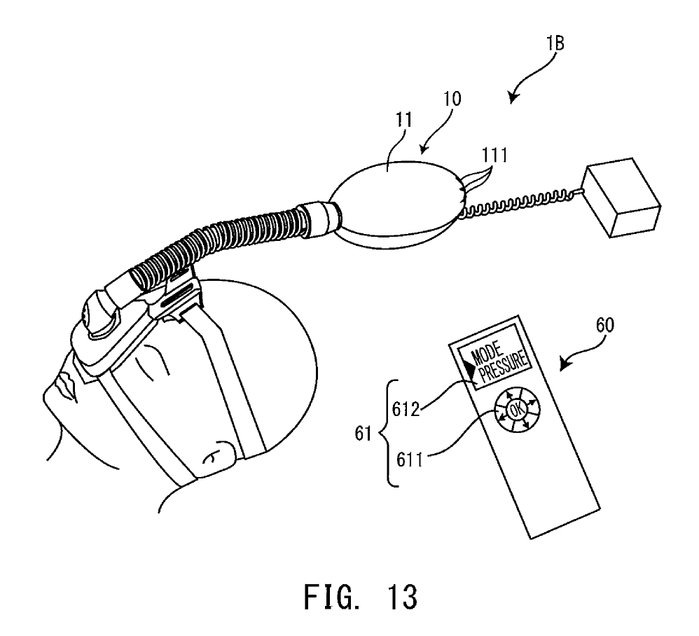

FIG. 13 is a view illustrating a usage condition of a CPAP apparatus according to a second embodiment.

Also in an air blowing unit 10 of an CPAP apparatus 1B illustrated in FIG. 13, plural air suction ports 111 are provided, on a side of the case 11 which side is away from the hose 20, similarly to the air blowing unit 10 of the CPAP apparatus 1A according to the first embodiment. On the other hand, in the air blowing unit 10 of the CPAP apparatus 1B illustrated in FIG. 13, the user interface 18 illustrated in FIG. 1 is not provided. Instead, in the CPAP apparatus 1B according to this second embodiment is provided with a remote controller 60. This remote controller 60 is provided with a user interface 61 including an operation button 611 and a display screen 612.

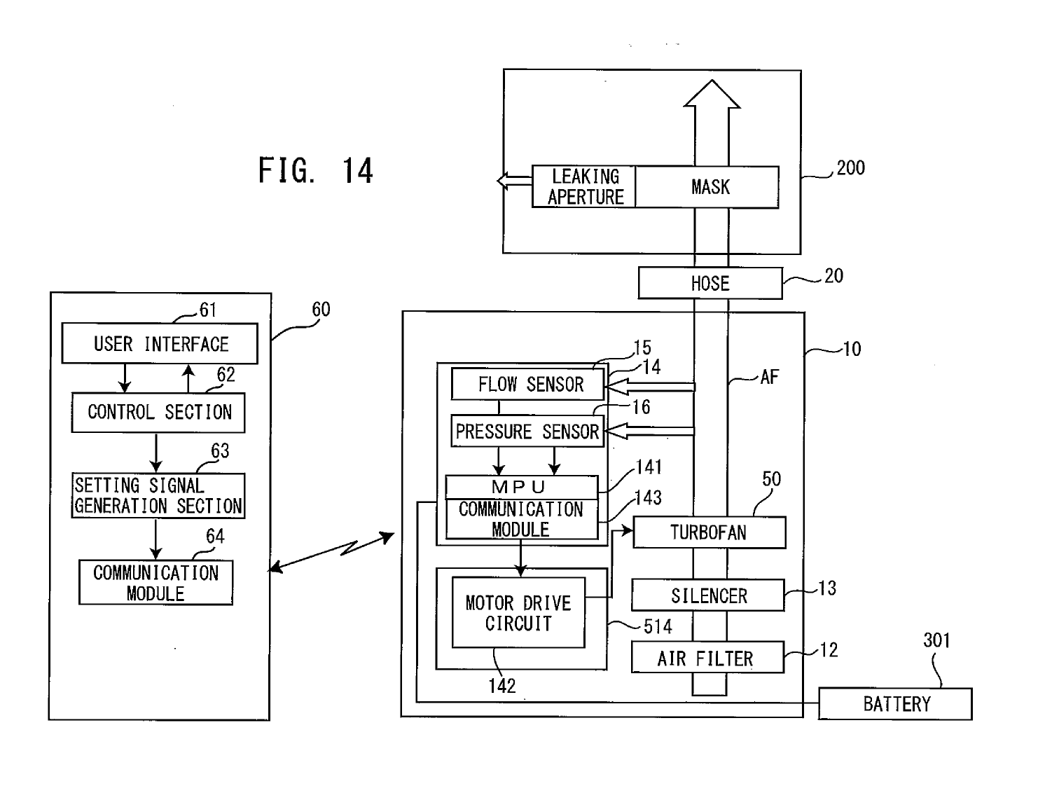

FIG. 14 is a block diagram of a control of the CPAP apparatus according to the second embodiment illustrated in FIG. 13. This FIG. 14 is a figure corresponding to FIG. 6 in the CPAP apparatus 1A according to the above-described embodiment.

The controller 60 illustrated in FIG. 14 is provided with the user interface 61 which is also illustrated in FIG. 13, and further, a control section 62, a setting signal generating section 63 and a communication module 64.

As illustrated in FIG. 13, the user interface 61 is composed of the operation button 611 which is operated by a doctor or a patient and the display screen 612 which gives information to the patient. With this user interface 61, similarly to the case of the user interface 18 included in the air blowing unit 10 of the CPAP apparatus 1A according to the above-described first embodiment, a selection between a fixed mode and an automatic mode, a pressure range of air, on-off timing of the turbofan 50 and the like are set by a doctor or a patient.

The control section 62 receives setting information of the user interface 61 and sends it to the setting signal generating section 63, and in addition, plays a role of controlling the display screen 612.

Compared to the air blowing unit illustrated in FIG. 6, the air blowing unit 10 in this FIG. 14 is different in that, the user interface 18 is removed, and a communication module 143 which performs wireless communication with the controller 60 is added.

Setting signals based on the information set in the user interface 61 are generated in the setting signal generating section 63. The generated setting signals are sent wirelessly to the air blowing unit 10 by the communication module 64 which performs wireless communication with the communication module 143 of the air blowing unit 10.

On the side of the air blowing unit 10, the setting signal sent from the remote controller 60 is received by the communication module 143, setting content by the setting signals is sent to the MPU 141. Subsequent processes are similar to those of the CPAP apparatus 1A according to the first embodiment which is described above, and overlapping explanations are omitted.

Next, alternative examples of the air suction port in the second embodiment will be explained.

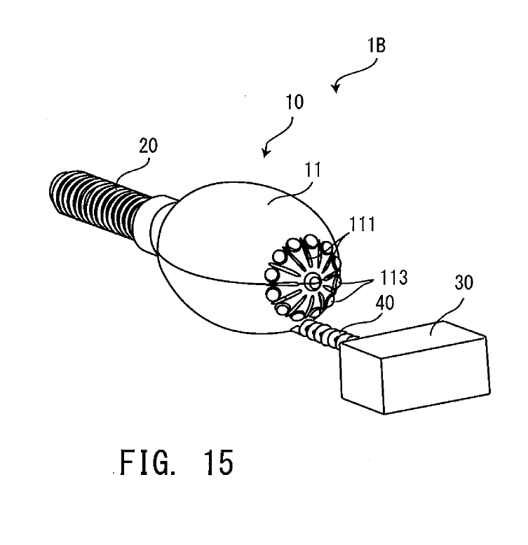

FIG. 15 is a view illustrating a first alternative example of the second embodiment.

Similarly to the first embodiment illustrated in FIG. 1 and the second embodiment illustrated in FIG. 13, plural air suction ports 111 which are arranged in a circumferential direction on a side away from the hose 20 are provided in the case 11 of the air blowing unit 10 illustrated in this FIG. 15.

Further, in this first alternative example illustrated in FIG. 15, projections 113 are provided between adjacent air suction ports 111. With these projections 113, even if something soft, for example, such as a cloth and a futon, covers over the air blowing unit 10, it is prevented that the air suction ports 111 are blocked.

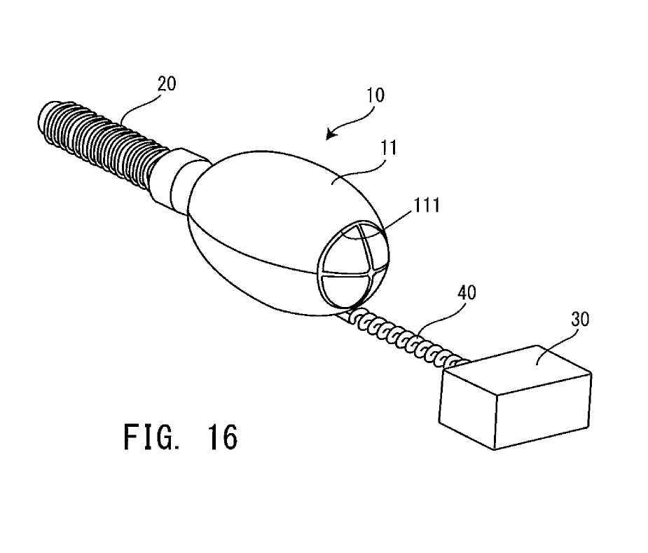

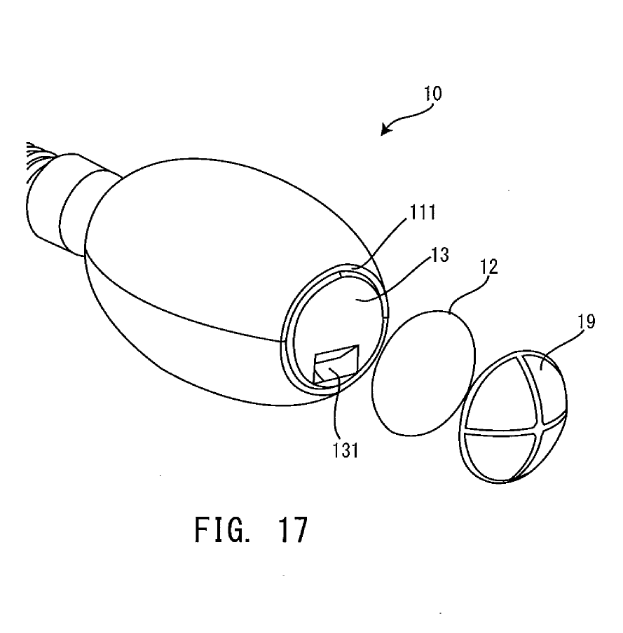

FIG. 16 is a view illustrating a second alternative example of the second embodiment. In addition, FIG. 17 is an exploded perspective view of an air inflow port of the second alternative example illustrated in FIG. 16.

An air suction port 111 having a large aperture is provided in the air blowing unit 10 of this second alternative example, and as illustrated in FIG. 17, it has a configuration in which when the air filter 12 and the like are removed, the silencer 13 inside the case 11 appears in the air suction ports 111. Air suctioned from the air suction port 111 is sent into the inside through an air flow path 131 of the silencer 13. An air filter 12 is attached to the air suction port 111, and the air filter 12 is covered by a surface filter member 19 which has a lot of holes in a mesh shape.

In this configuration, since air can be taken in from all over a whole area of the surface filter member 19, it is prevented that air suction is disturbed in an normal usage condition.

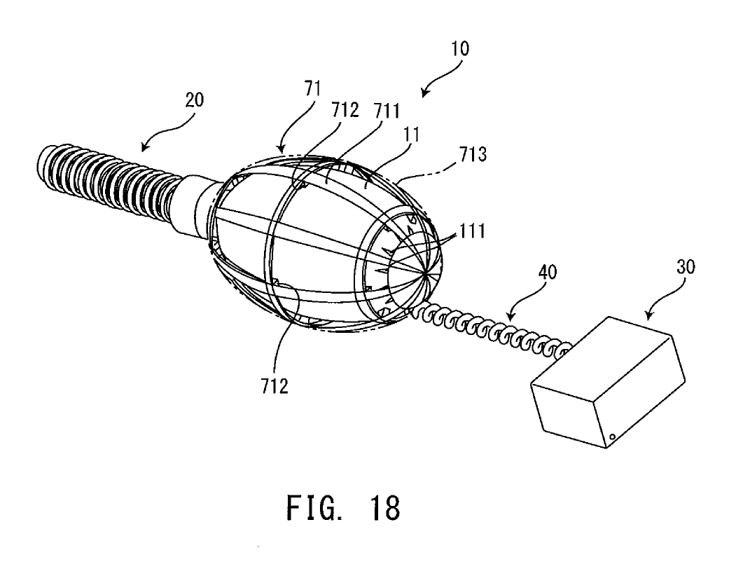

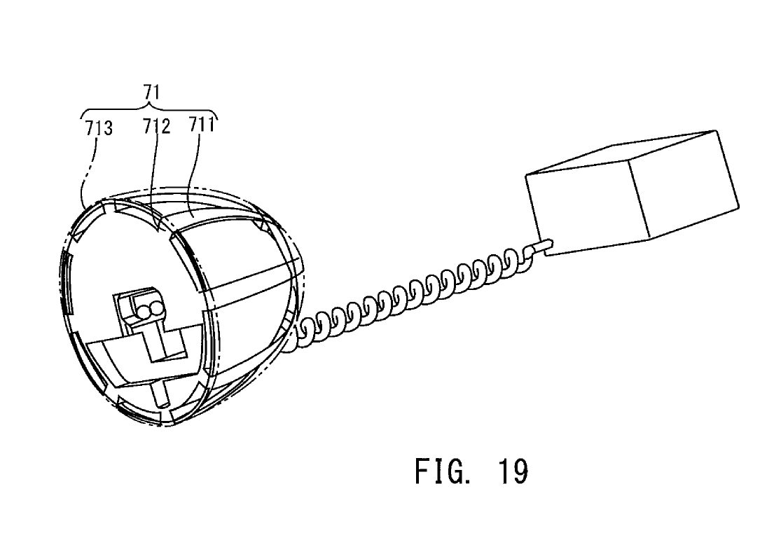

FIG. 18 is a view illustrating a third alternative example of the second embodiment. In addition, FIG. 19 is a sectional perspective view of an air blowing unit of the third alternative example illustrated in FIG. 18.

In FIG. 18, a case 11 and plural air suction ports 111 provided in a case 11 are illustrated while being seen through a external shell case outer sheath 713 covering around them.

An external shell case 71 is a member corresponding to an example of the guard section according to the present invention. In this external shell case 71, some portions of an external case skeleton 711 which is arranged at a position away from the case 11 are supported by an external case stay 712, and further, the external shell case outer sheath 713 which is made of sponge-like porous material is arranged such that the external shell case outer sheath 713 covers the external case skeleton 711. This external shell case outer sheath 713 allows air to be suctioned through whichever part thereof, and the suctioned air flows along a surface of the case 11 to be suctioned to the inside from the air suction port 111 of the case 11.

Also with the configuration of the CPAP apparatus according to this third embodiment, an event that air suction is disturbed by a cloth, a futon or the like is prevented.

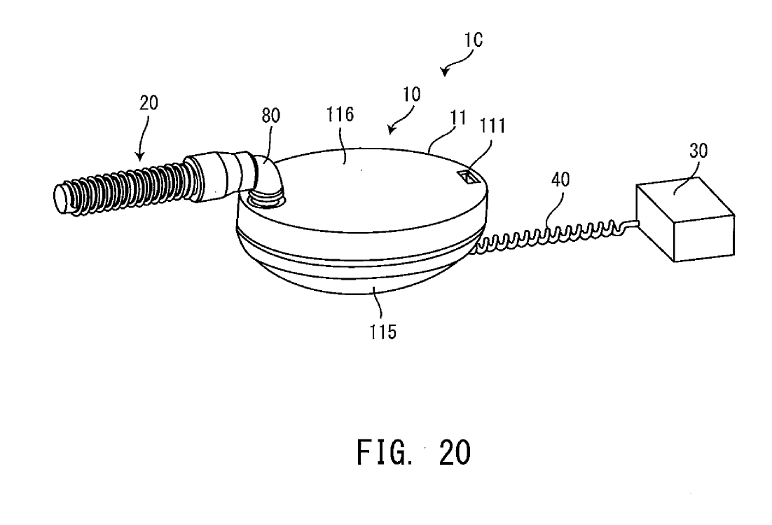

FIG. 20 is an exploded perspective view of a CPAP apparatus according to a third embodiment.

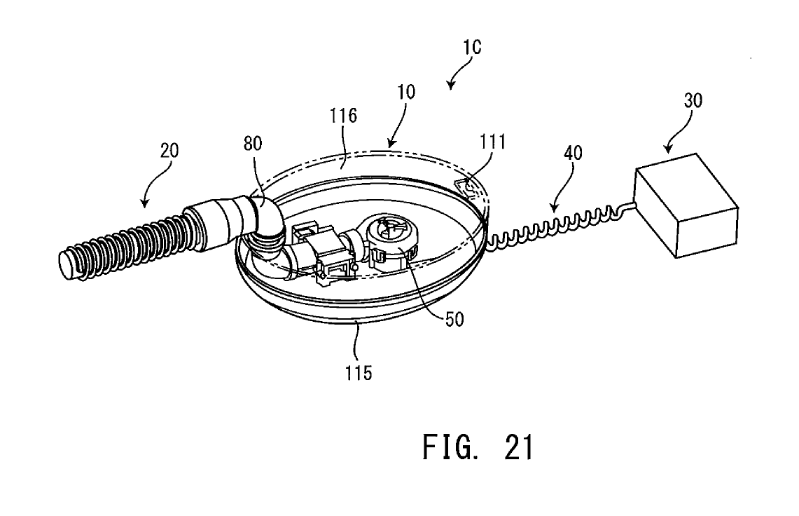

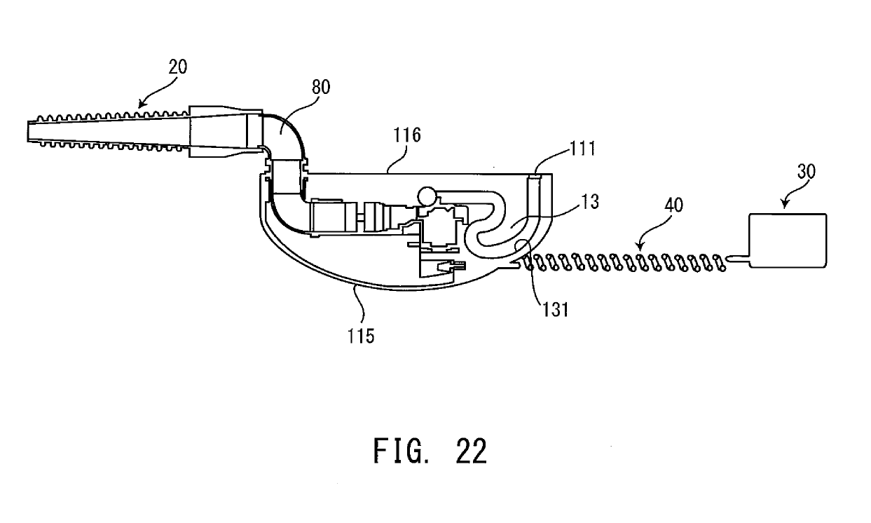

In addition, FIG. 21 is a transparent perspective view illustrating an air blowing unit of the CPAP apparatus according to the third embodiment illustrated in FIG. 20. However, in this FIG. 21, illustration of the silencer is omitted. Further, FIG. 22 is a sectional view of the air blowing unit of the CPAP apparatus according to the third embodiment.

A case 11 of an air blowing unit 10 of a CPAP apparatus 1C according this third embodiment includes a boat-form bottom surface 115 which is formed in a convex surface such as an oval surface and the like, and an upper surface 116 which is approximately flat. An air suction port 111 is arranged in the upper surface 116.

Air suctioned from this air suction port 111 included in the silencer 13 flows through an air flow path 131 which curves as illustrated in FIG. 22, and is blown out by the turbofan 50.

This air blowing unit 10 is connected to the hose 20 via a movable joint 80. This movable joint 80 is a joint which is rotatable around a perpendicular axis.

In the case of this third embodiment, heavy components such as the turbofan 50 are arranged in a bottom portion of the case 11, and the silencer 13 which is lightweight and large in size is arranged in an upper portion inside the case 11. For this reason, this air blowing unit 10 is made such configured that the air blowing unit 10 is scarcely turned upside down even when it sways or moves.

The air blowing unit 10 of the CPAP apparatus 1C according to this third embodiment is used while being place bedside, similarly to the air blowing units 1A, 1B according to the first embodiment and the second embodiment which are described above.

When a patient in a lying posture while wearing a mask 200 (see FIG. 2) on its face moves, a force is applied to the air blowing unit 10 via the hose 20 in association with the moving, and at this moment, the air blowing unit 10 firstly follows with the movable joint 80, and when a force is further applied, it moves in a swinging motion with the boat-form bottom surface 115, and when a force is furthermore applied, the air blowing unit 10 slides to follow the moving of the patient.

As explained, the air blowing unit of the CPAP apparatus according to the present invention may be configured to roll, or may be configured to slide to follow moving of a patient.

In the foregoing, the types in which the air blowing unit is formed in an oval spherical shape and it is supposed to mainly roll (the first embodiment and the second embodiment) and the type in which the air blowing unit has a boat-form bottom surface and it is supposed to swing or slide (the third embodiment) have been explained, and in the following, embodiments including air blowing units having external appearances different from those described above will be explained. Incidentally, in each of the drawings which will be explained in the following, illustration of the battery case 30 and the cable 40 is omitted.

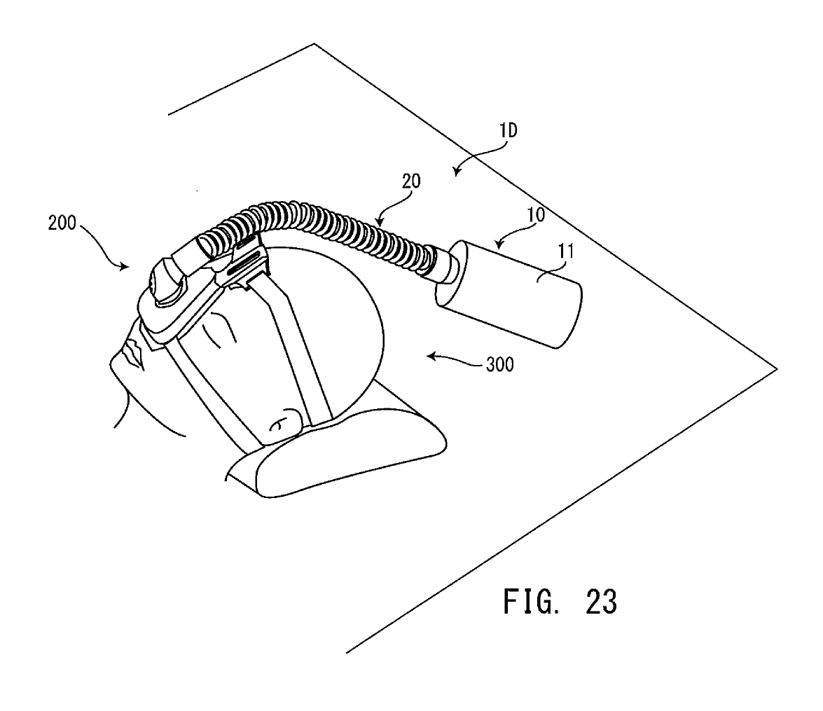

FIG. 23 is a view illustrating a usage condition of a CPAP apparatus according to a fourth embodiment.

An air blowing unit 10 of a CPAP apparatus 1D according to the fourth embodiment illustrated in here includes a cylindrically-shaped case. Also in a case in which an air blowing unit 10 including such a cylindrically-shaped case is provided, it is possible to allow the air blowing unit 10 to easily roll in accordance with a posture of a patient.

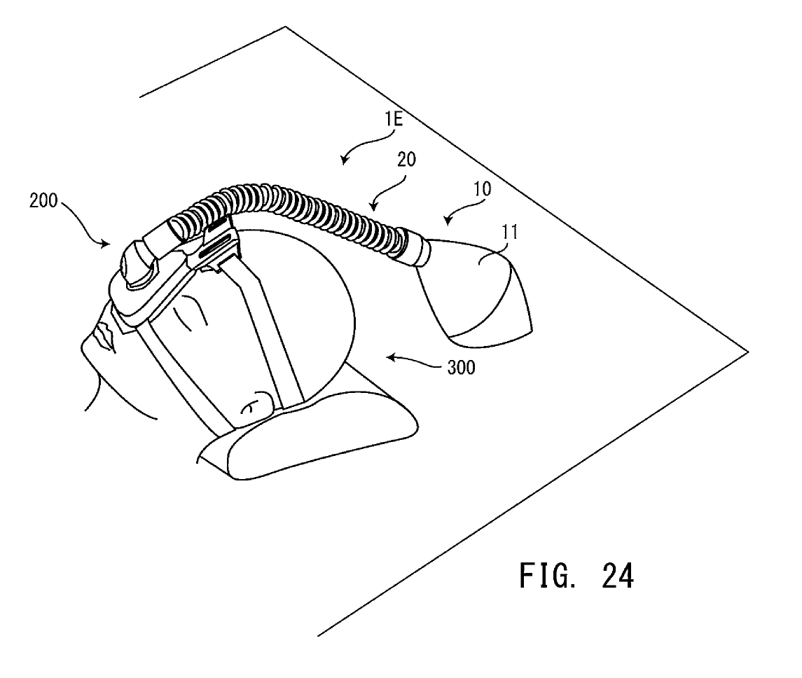

FIG. 24 is a view illustrating a usage condition of a CPAP apparatus according to a fifth embodiment.

An air blowing unit 10 of a CPAP apparatus 1E according to the fifth embodiment illustrated here includes a case having a shape in which two cones are attached with each other. Also in a case in which the air blowing unit 10 includes such a cone-shaped case, it is possible to allow the air blowing unit 10 to roll easily in accordance with a posture of a patient Incidentally, as far as only a side away from the hose 20 has a cone shape, it does not matter what a shape on a side near the hose 20 is.

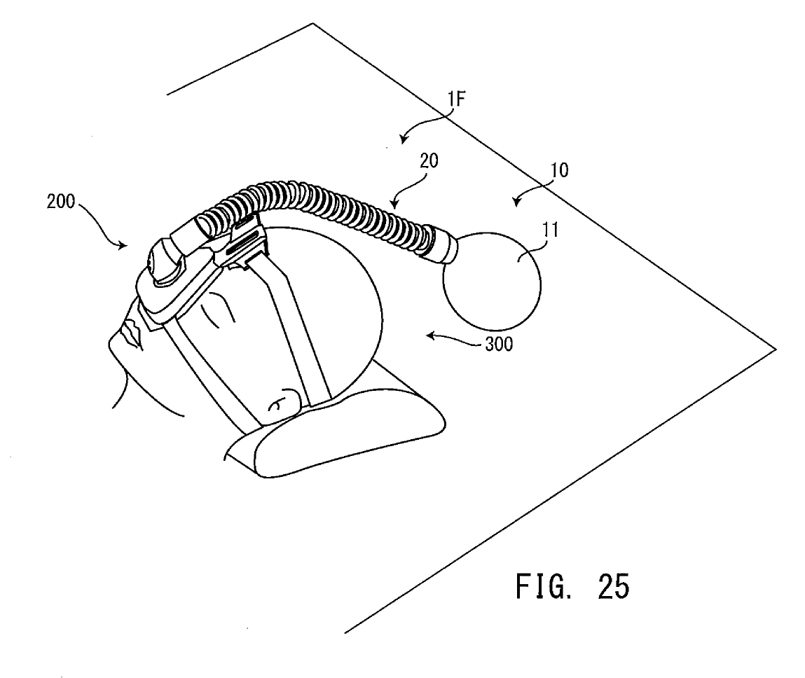

FIG. 25 is a view illustrating a usage condition of a CPAP apparatus according to a sixth embodiment.

An air blowing unit 10 of the CPAP apparatus 1F according to the sixth embodiment illustrated in here includes a case 11 having a spherical shape. In a case in which the air blowing unit 10 including such case having a spherical shape is provided, it is possible to allow the air blowing unit 10 to easily roll according to a posture of a patient.

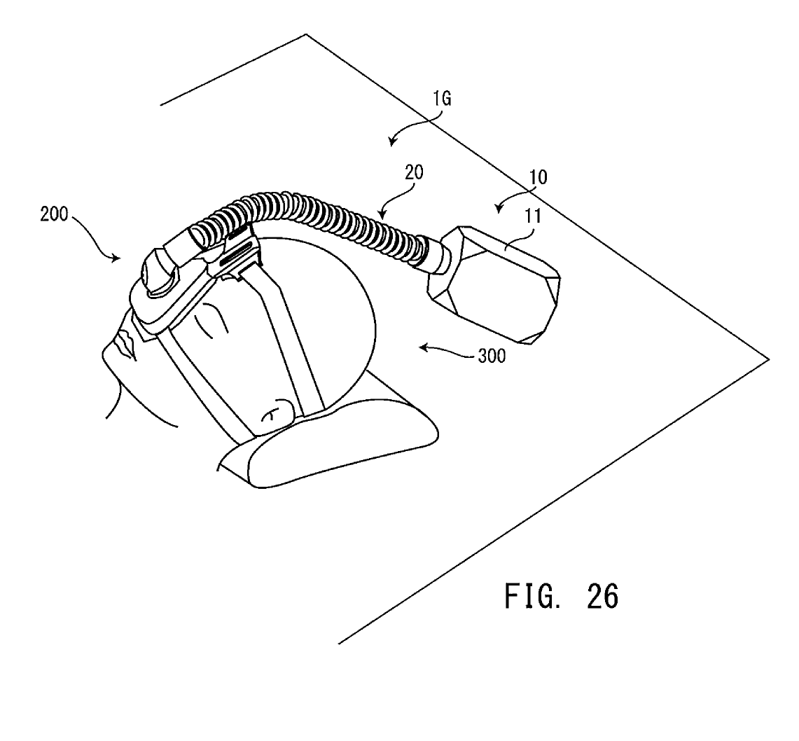

FIG. 26 is a view illustrating a usage condition of a CPAP apparatus according to a seventh embodiment.

An air blowing unit 10 of a CPAP apparatus 1G according to the seventh embodiment illustrated in here includes a case 11 having a shape in which eight corners of a square pole are beveled to include fourteen surfaces in total. Also in a case in which such air blowing unit 10 whose case includes such a pseudo curved surface is provided, it is possible to allow the air blowing unit 10 to easily roll in accordance with a posture of a patient.

Each of the above-described embodiments is a type in which the air blowing unit 10 is placed on a floor on a patient's bedside, and however, the air blowing unit according to the present invention may be one as far as it is supported by other than the hose 20 at a position away from a patient in a lying posture, the hose follows posture changing of the patient while being in a lying posture, and the air blowing unit receives a force via the hose 20 to change a position or a posture thereof.

For example, not limited to a case in which it is placed on a bed or a futon, it may be an air blowing unit which has a configuration in which the air blowing unit is hung at a bed side by using an attaching part and changes a position or a posture thereof according to a posture of a patient. Or, the air blowing unit 10 may be placed on a breast or a place thereabout of a patient in a lying posture.

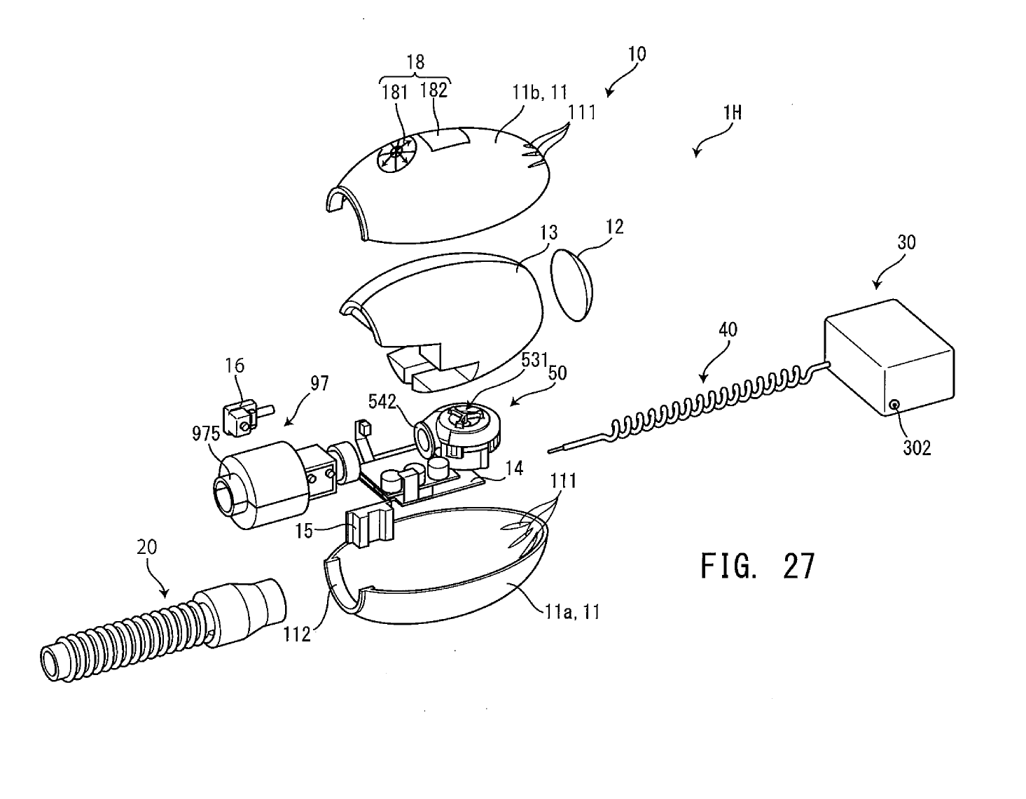

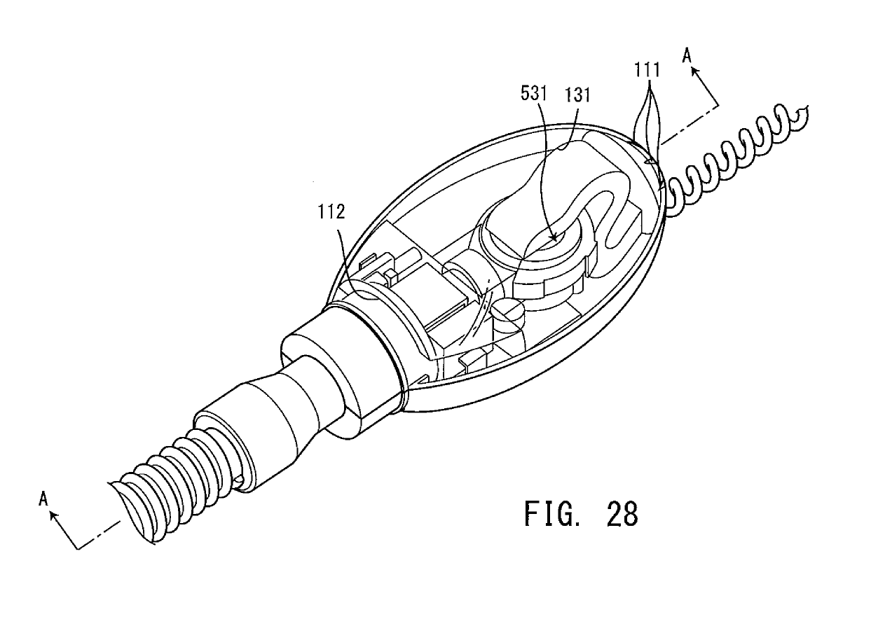

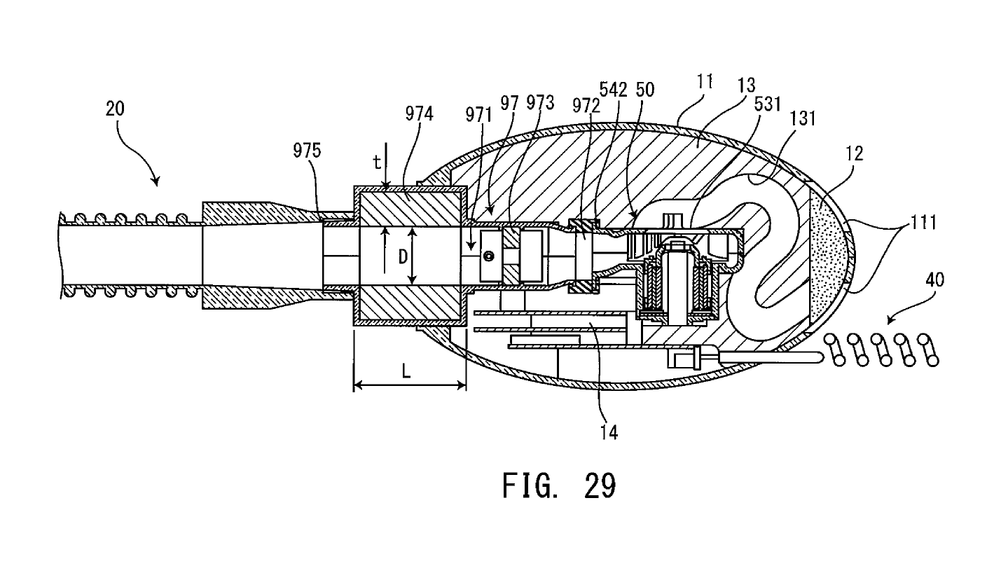

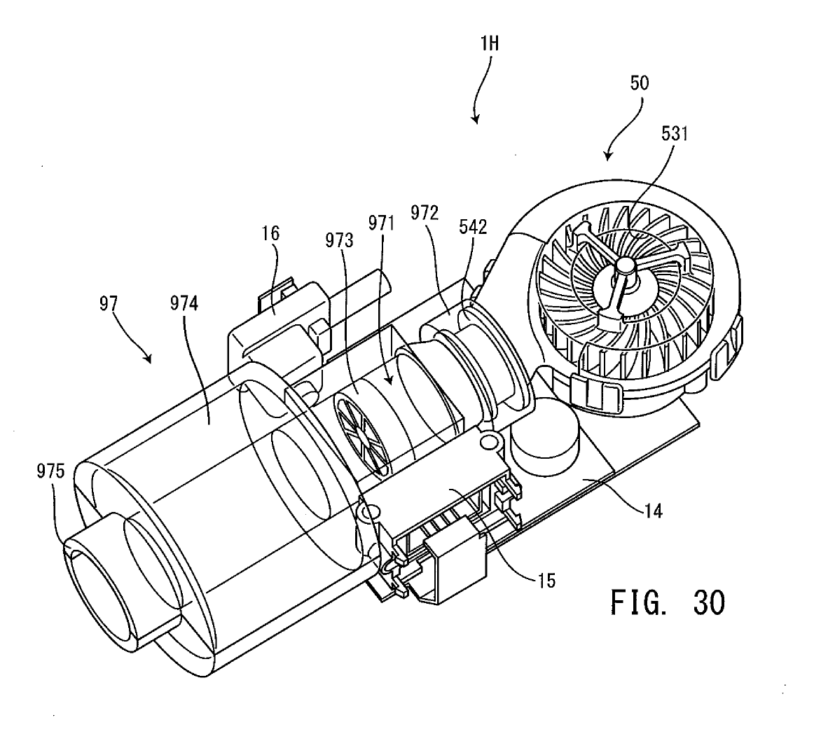

FIG. 27 is an exploded perspective view illustrating a CPAP apparatus according to a eighth embodiment. In addition, FIG. 28 is a transparent view when the CPAP apparatus according to the eighth embodiment is viewed obliquely from above, and FIG. 29 is a sectional view along arrows A-A illustrated in FIG. 28 of the CPAP apparatus according to the eighth embodiment. Further, FIG. 30 is a transparent view when the case and the suction silencer are removed from the CPAP apparatus according to the eighth embodiment, and a fan, a discharge structure body and the like are viewed obliquely from above.

Incidentally, external appearances and how to use of a CPAP apparatus 1H according to this eighth embodiment are similar to those in FIG. 1 and FIG. 2 illustrated for the CPAP apparatus 1A according to the first embodiment, and overlapped illustration and explanations will be omitted here.

In the CPAP apparatus 1H according to the eighth embodiment, a case 11 of an air blowing unit 10 is configured by a case lower section 11a and a case upper section 11b.

Since this case 11 is made to have an oval shape as a whole, it easily rolls. In addition, this case 11 is made of plastic and an external surface is formed to be smooth and thus easily move slidably. In order that suctioning of air is not disturbed even when this case 11 rolls or slides, plural air suction ports 111 are provided in the case 11.

In addition, in the case upper section 11a, there is provided a user interface 18 including an operation button 181 and a display screen 182.

In the case 11, there are arranged an air filter 12, a suction silencer 13, a control board 14, a flow sensor 15, a pressure sensor 16, a discharge silencer 97 and a turbofan 50 as the fan.

In addition, as described above, in the CPAP apparatus 1H, there are provided the hose 20, the battery case 30 and the cable 40.

The air filter 12 is a filter which is arranged immediately inside the air suction ports 111 provided in the case 11, and absorbs dusts in air suctioned from the air suction ports 111.

In addition, the suction silencer 13 has a suction path 131 which is curved as illustrated in FIG. 4 and FIG. 5, and guides the air suctioned form the air suction ports 111 to an air receiving port 531 of the turbofan 50. This suction silencer 13 plays a role of decreasing suction sound of the air suctioned from the air suction ports 111 to introduce the air to the turbofan 50. In addition, this suction silencer 13 also plays a role of supporting the turbofan 50 such that the suction silencer 13 enfolds the turbofan 50 by the sound absorbing member, and preventing vibrations of the turbofans 50 from being transmitted to the case 11 or other members.

The turbofan 50 causes air to be suctioned from the air suction ports 111 of the case 11, receives from the air suction port 531 the air coming via the air filter 12 and the air suction silencer 13 and sends out the air from the air blowing port 542.

The control board 14 calculates a rotation setting speed of the turbofan 50 according to an initial setting by a doctor or a patient, flow amounts measured by the flow sensor and pressures measured by the pressure sensor 16, and gives an instruction to the turbofan 50 to rotate at the rotation speed.

The flow sensor 15 and the pressure sensor 16 are sensors which measure flow amounts and pressures of the air sent out from the turbofan 50, respectively.

The discharge silencer 97 is coupled to the air blowing port 542 of the turbofan 50 to form a discharge path 971, and allows the air sent out from the air discharge port 542 by the turbofan 50 to be emitted from this air blowing unit 1H. Between this discharge silencer 97 and the air blowing port 542 of the turbofan 50 is connected with a joint 972 made of rubber. This joint 972 plays a role of preventing that vibrations of the turbofan 50 are transmitted to the discharge silencer 97 to increase noises.

In this discharge silencer 97, there are provided a rectifying element 973 and a sound absorbing member 974. The rectifying element 973 is a member to play a role of rectifying a flow of air sent in from the turbofan 50. The flow sensor 15 and the pressure sensor 16 are connected to a downstream side with respect to the flow of the air of the rectifying element 973. With this, it is prevented that an unnecessary change by air turbulence is transmitted to the flow sensor 15 or the pressure sensor 16 so that measured values of the air flow or the air pressure are unnecessarily changed.

In addition, the sound absorbing member 974 plays a role of reducing noise as the air flows which air is sent out from the air blowing port 542 by the turbofan 50. This sound absorbing member 974 is a sound absorbing member made of foamed material, for example, urethane foam or EVA (Ethylene Vinyl Acetate) foam. The density of the foamed material is preferably to be within a range of 10 to 100 k g/m³.

The sound absorbing member 974 provided in the suction silencer 97 effectively decreases noises as a patient breathes, as indicated in experimental data which will be explained later. The hose 20 is coupled to an air discharge port 975 of the discharge silencer 97, air is sent into the mask 200 via the hose 20.

A battery 301 is housed inside the battery case 30, and electrical power from the battery 301 is supplied to the air blowing unit 10 via the cable 40. This battery case 30 is provided with a connecting terminal 302 to which an AC adapter (not illustrated) which charges an inside battery is connected. A battery is a component having a significant volume and a significant weight, and in order to make the air blowing unit 10 compact and lightweight, in here, a configuration in which the battery case 30 which is separate from the air blowing unit 10 is provided with and is connected by the cable 40 is applied. However, a configuration in which the battery case 30 and the large battery 301 are not provided and an AC adapter is connected to the air blowing unit 10 to allow the air blowing unit 10 to operate may be applied.

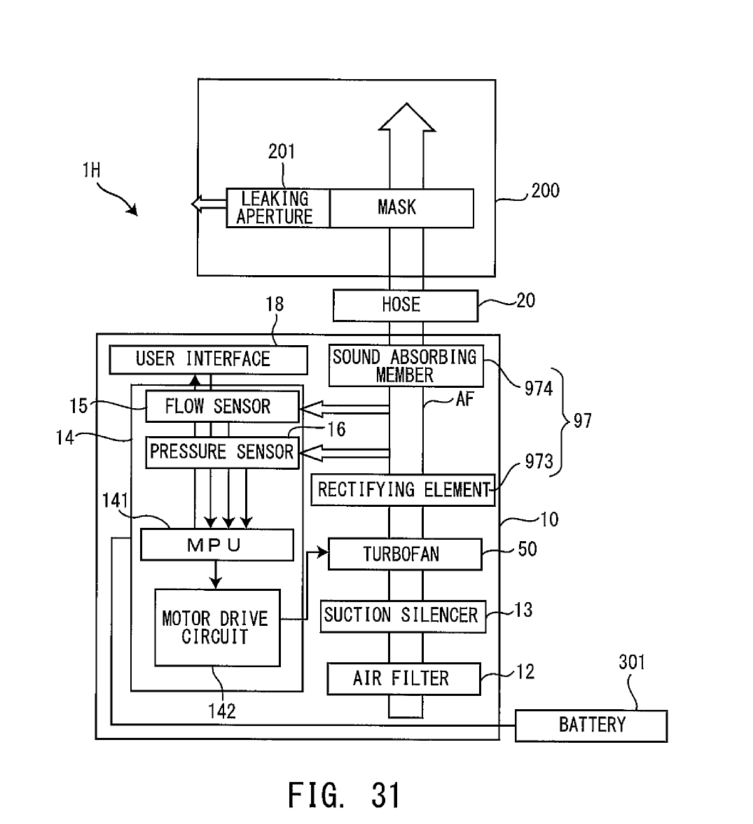

FIG. 31 is a control block diagram of the CPAP apparatus according to the eighth embodiment.

In here, an air flow path AF which flows from an air blowing unit 10 via a hose 20 to a mask 200 and a control system of the air blowing unit 10 are illustrated.

As described above, in the air blowing unit 10, an air filter 12, a suction silencer 13, a turbofan 50 and a rectifying element 973 and a sound absorbing member 974 which element 973 and member 974 are included in a discharge silencer 97 are arranged on the air flow path AF. When the turbofan 50 rotates, air is suctioned from the air suction ports 111 (see, for example, FIG. 28), dusts in the air are removed by the air filter 12, noises as the air is suctioned are reduced by the suction silencer 13, through the turbofan 50, further the air is regulated by the rectifying element 173, furthermore noises are reduced by the sound absorbing member 974 and the air is sent into the mask 200 via the hose 20.

The air sent into the mask 200 is sent into a respiratory tract of a patient, and is discharged through a leaking apertures 201 to the outside by expiration of the patient.

This air blowing unit 10 is provided with a user interface 18 including an operation button 181 and a display screen 182 (see, for example, FIG. 1). A patient operates the operation button 181 while checking the display screen 182, to set a selection between a fixed mode and an automatic mode, a pressure range of air sent out from the turbofan 50 which pressure range is designated from a doctor, on-off timing of the turbofan 50 and the like. Here, the fixed mode is a mode in which a pressure of air sent out from the turbofan 50 is fixed to a designated pressure, and the automatic mode is a mode in which a breathing state of a patient is detected from changes of flow amounts or pressures by the flow sensor 15 or the pressure sensor 16, the pressure is changed in the designated range according to the breathing state of the patient.

Information set by the user interface 18 is input into an MPU (Micro Processing Unit) 141. In addition, air flow amounts and air pressures measured by the flow sensor 15 and the pressure sensor 16 are also input into the MPU 141. The MPU 141 calculates a rotation speed of the turbofan 50 based on those pieces of the information. A result of the calculation by the MPU 141 is sent to the motor drive circuit 142, and the motor drive circuit 142 drives the turbofan 50 based on the result of the calculation.

The flow sensor 15, the pressure sensor 16 and the MPU 141 are mounted on the control board 14 (see, for example, FIG. 27) included in the air blowing unit 10. Electrical power is supplied to the control board 14 from the battery 301, and electrical power is distributed to each of sections which require the electrical power. In addition, in the present embodiment, the motor drive circuit 142 is also mounted on the circuit board 14.

One of characteristics of the CPAP apparatus according to the present embodiment is in that the turbofan 50 provided with the air dynamic bearing as one example of the fluid dynamic bearing is applied, similarly to each of the CPAP apparatuses 1A to 1G according to the above-described embodiments. Thanks to this, the CPAP apparatus 1H according to the present embodiment succeeds in making the air blowing unit 10 downsized and lightweight significantly.

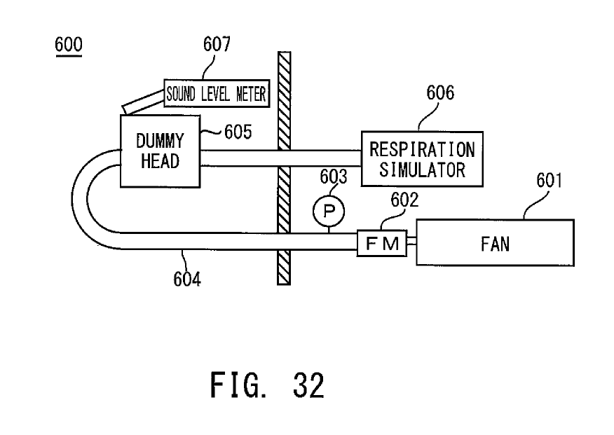

FIG. 32 is a schematic diagram of an experimental equipment.

A dummy head 605 which mimics a shape of a human head and is worn with a mask is placed in an anechoic room 600, and between a fan 601 which is placed outside the anechoic room 600 and the dummy head 605 is coupled by a hose 604 having a length of approximately 2.5 meters. A flow meter 602 and a manometer 603 are placed at an air output port of the fan 601 and flow amounts and pressures are measured. In addition, a respiration simulator 606 is coupled to the dummy head 605. This respiration simulator 606 has a function to simulate inspiration and expiration and corresponds to a human lung, and a noise meter 607 is provided near the dummy head 605 (a position corresponding to a human ear), noises when respiration simulations are performed by the respiration simulator 606 are measured.

Here, as the fan 601, a fan (blade diameter: approximately 53 mm, weight: approximately 240 g) (Hereafter, this fan will be referred to as “Fan of Comparable Example” or simply “Comparable Example”) which is incorporated in a commonly commercially available stationary CPAP apparatus, and a fan (blade diameter: 29 mm, weight: approximately 40 g) (hereafter, this fan will be referred to as “Fan of Embodiment Example” or simply “Embodiment Example”) which is equivalent to the turbofans used in the embodiments are used. Basically, the Fan of Embodiment Example is a fan of air dynamic bearing, which is explained above with reference to FIGS. 7 to 12.

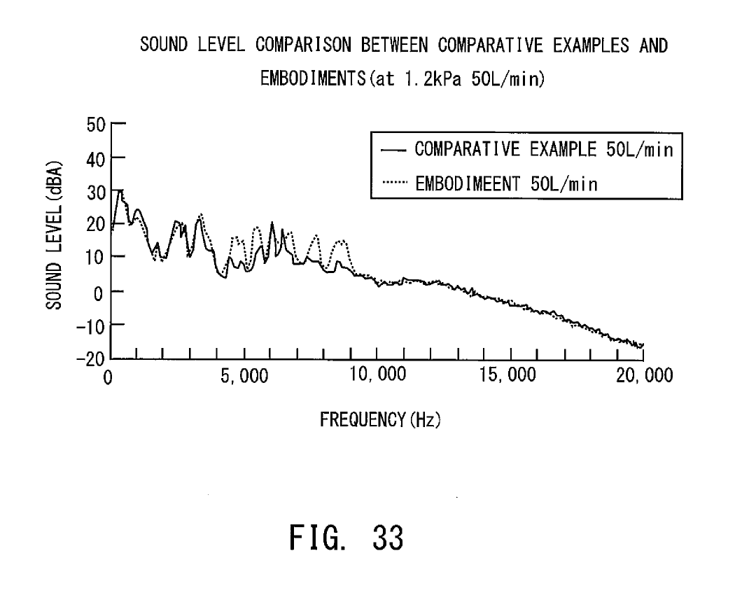

FIG. 33 is a view illustrating noises of fans of a comparative example and an embodiment when the pressure is 1.2 kPa and the flow amount of flowing is 50 L/min (litter/minute). However, the “Fan of Embodiment Example” is a fan only which is not provided with a silencer. The horizontal axis represents frequencies (Hz), and the vertical axis represents noise levels (dBA). The flow amount of 50 L/min corresponds to a time when breathing stops (a time between an expiration and an inspiration). When noise in order of 5 kHz to 7 kHz are large, the noise are tend to be easily sensed as being harsh to one's ears, and it is required to reduce noise of such frequency band. Looking into the noise levels at 5 kHz to 7 kHz, at Pressure 1.2 kPa and Flow Amount 50 L/min (breathing stops) as illustrated in this FIG. 15, the noises of the Embodiment Example are slightly larger than those of the Comparative Example.

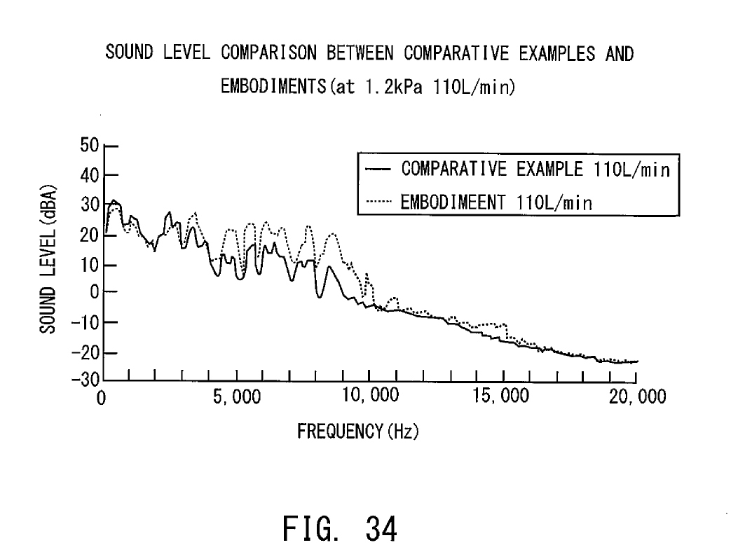

FIG. 34 is a view illustrating noises of fans of the comparative example and the embodiment when the pressure is 1.2 kPa and the flow amount is 110 L/min. The Pressure 1.2 kPa and the Flow Amount 110 L/min correspond to a time of inspiration. Also in here, the “Fan of Embodiment Example” is a case in which the fan is not provided with a silencer and is a fan only.

At Pressure 1.2 kPa, Flow Amount 110 L/min as illustrated in this FIG. 34, the noises of the Fan of Embodiment Example are larger than those of the Fan of Comparative Example. In the sense of hearing, a ‘shoo’ sound is heard at the time of inspiration.

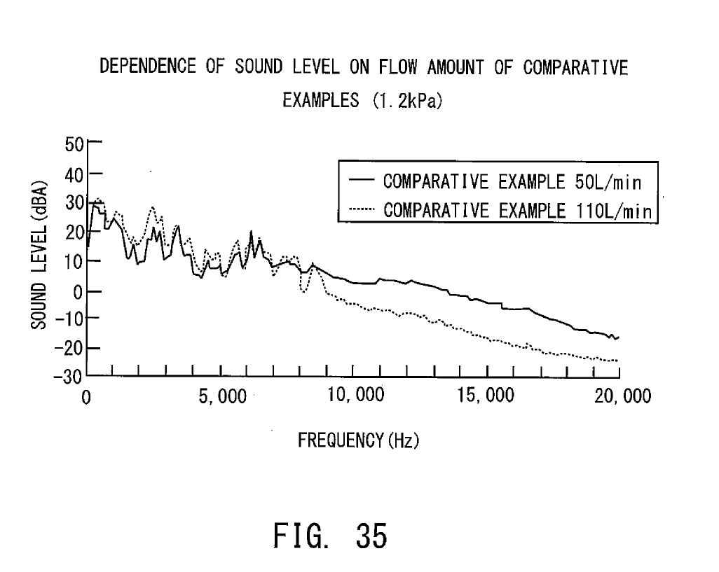

FIG. 35 is a view illustrating noises of the fan of the comparative example at the time when breathing stops and at the time of inspiration.

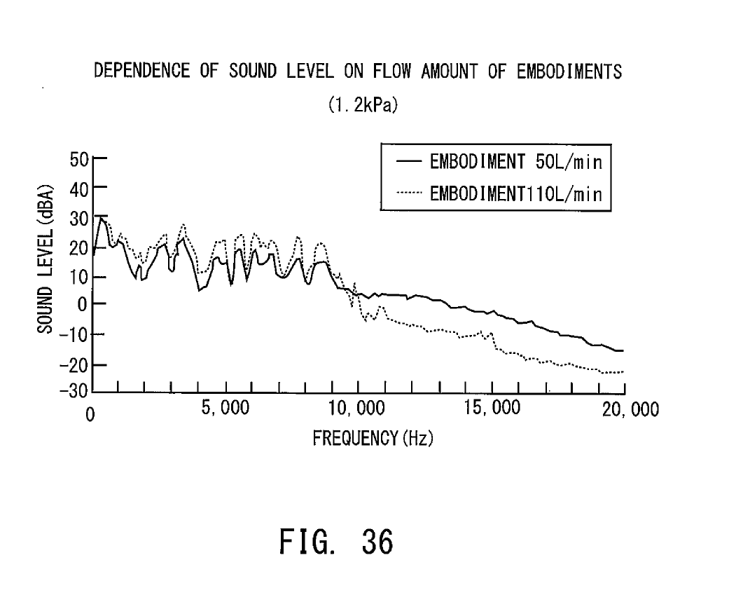

In addition, FIG. 36 is a view illustrating noises of the fan of the embodiment at the time when breathing stops and at the time of inspiration.

Comparing FIG. 35 with FIG. 36, it is found that, with respect to about 5 kHz to 7 kHz, increased amounts of the noises when at the time of inspiration compared to those at the time of breathing stop are larger in FIG. 36 (Fan of Embodiment Example) than those in FIG. 35.

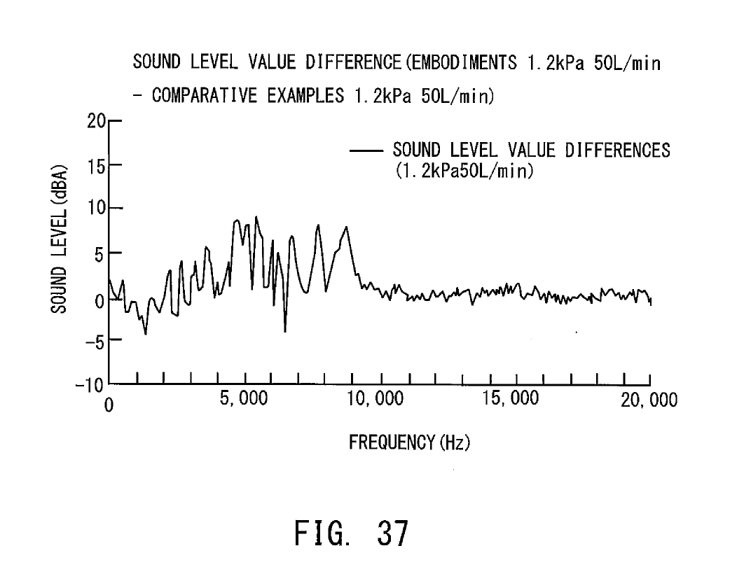

FIG. 37 is a view illustrating differences between noise levels of the fan of the embodiment and the noise level of the fan of the comparative example at the time when breathing stops. In other words, this FIG. 37 illustrates differences of the two graphs illustrated in FIG. 33.

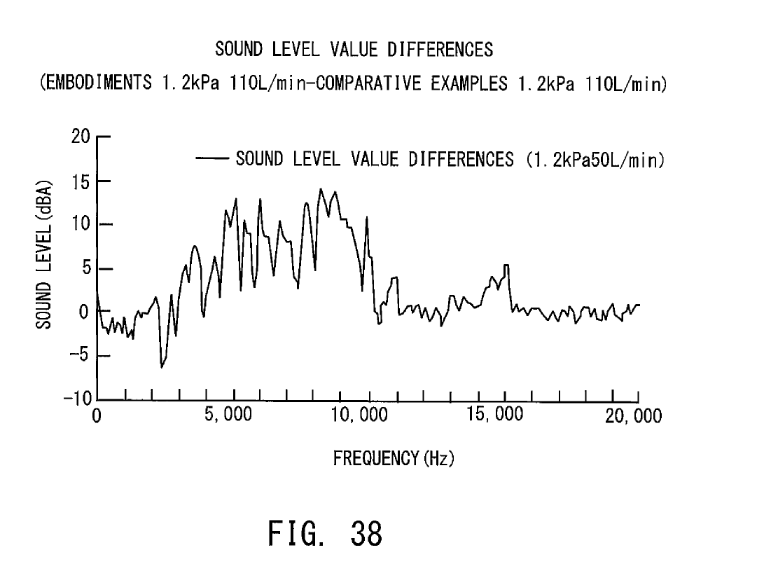

In addition, FIG. 38 is a view illustrating differences between noise levels of the fan of the embodiment and noise levels of the fan of the comparative example at the time of inspiration. In other words, this FIG. 38 illustrates differences of the two graphs illustrated in FIG. 34.

As found from these FIG. 37 and FIG. 38, it is found that, at both of at the time when breathing stops (FIG. 37) and at the time of inspiration (FIG. 38), the noises of the Fan of Embodiment Example are larger than those of the Fan of Comparative Example, and specifically at the time of inspiration (FIG. 38).

If the fan of the Embodiment Example is applied, compared to a conventional CPAP apparatus in which the Fan of Comparative Example is applied, downsizing and reducing in weight are achieved significantly, and however, as explained above, in terms of noise, it becomes disadvantageous largely. This is because it is required to send air of a flow amount same as that of the Fan of Comparative Example to cause the fan to rotate faster according to that the fan of the embodiment is smaller. In addition, it also becomes an disadvantageous factor that the changes of the rotation speed of the fan with respect to the changes of the flow amount becomes large.

Accordingly, next, experimental data in a case in which a discharge silencer is attached at a side of an air blowing port of the Fan of Embodiment Example will be introduced.

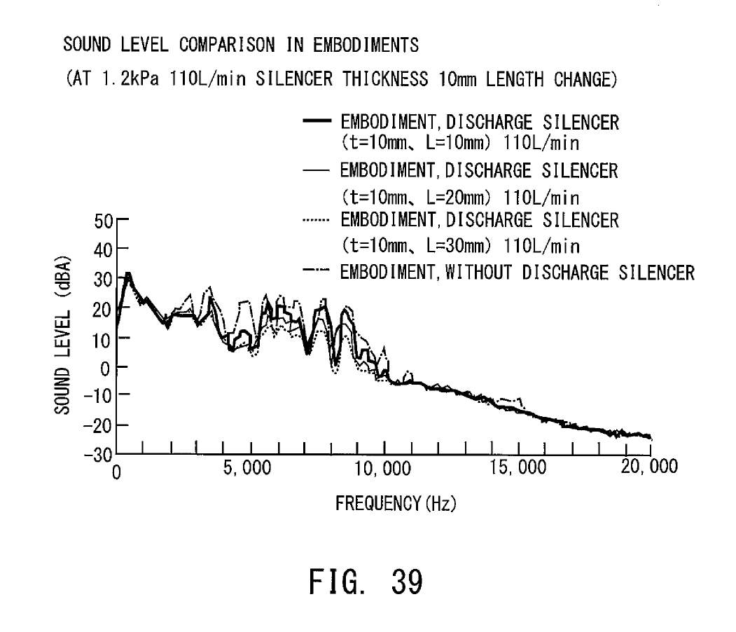

FIG. 39 is a view illustrating changes of noise levels when a length of the discharge silencer is changed at the time of inspiration.

Here, urethane foam is used as the sound absorbing member. The thickness illustrated in FIG. 29 is t=10 mm, and in this FIG. 39, noise levels when the length L is made to three types of L=10 mm, 20 mm and 30 mm illustrated in FIG. 29 are illustrated. In addition, in this FIG. 39, noise levels when a discharge silencer is not applied (see FIG. 34) are also illustrated. The diameter D of the discharge path is D=12 mm.

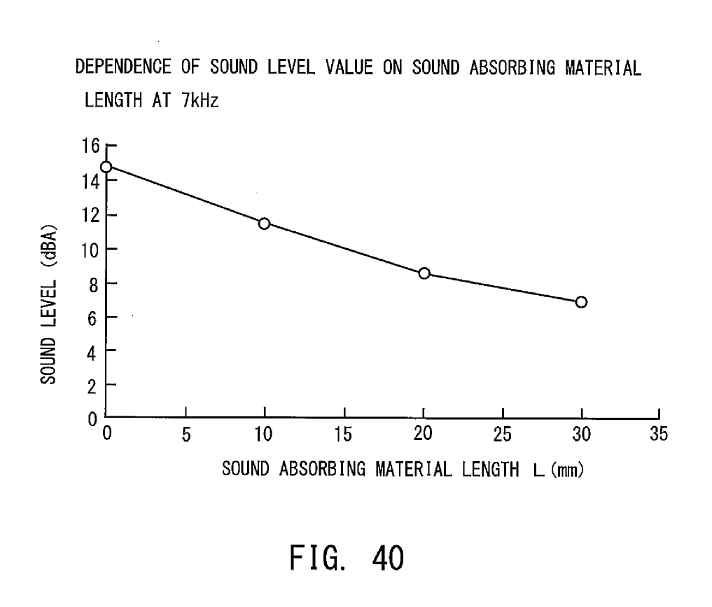

FIG. 40 is a view illustrating noise levels at 7 kHz with respect to the length of the sound absorbing member included in the discharge silencer which noise levels are read and obtained from the FIG. 39.

AS found in FIG. 39 and FIG. 40, the longer the length of the sound absorbing member is, the larger the effects of absorbing the noise become, and thus the noise levels are decreased. More specifically, under the experimental condition illustrated in FIG. 32, when a discharge silencer having a length of the order of L=20 mm is provided, it possible to reduce noises more compared to the Fan of Comparative Example.

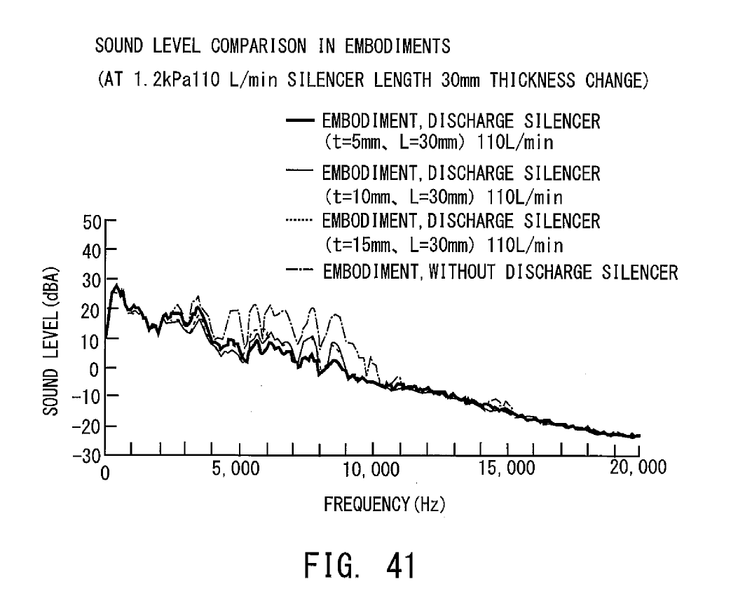

FIG. 41 is a view illustrating changes of noise levels when the thickness of the discharge silencer is changed at the time of inspiration.

As the sound absorbing member, urethane foam is applied as same as the case of FIG. 39. Here, the length L of the sound absorbing member is fixed to L=30 mm, and the thickness t is changed to t=5 mm, 10 mm and 15 mm. In addition, sound levels when a discharge silencer is not applied are also illustrated in here.

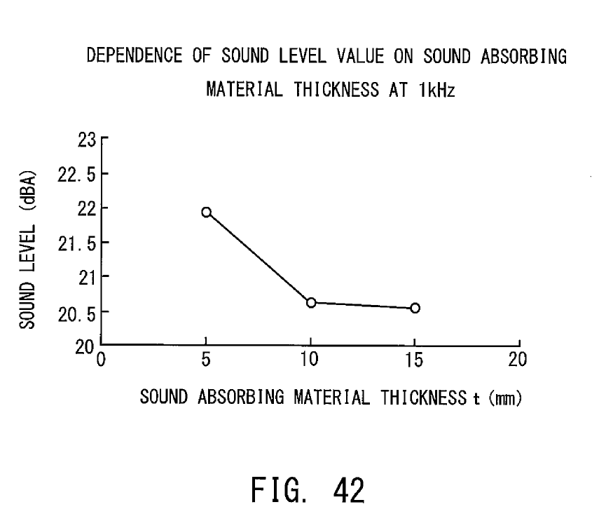

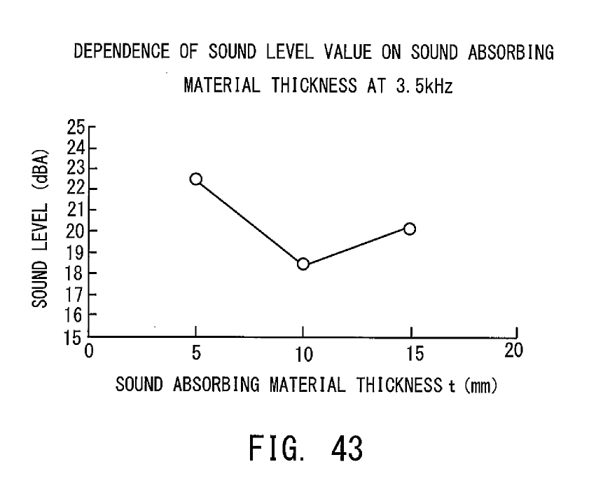

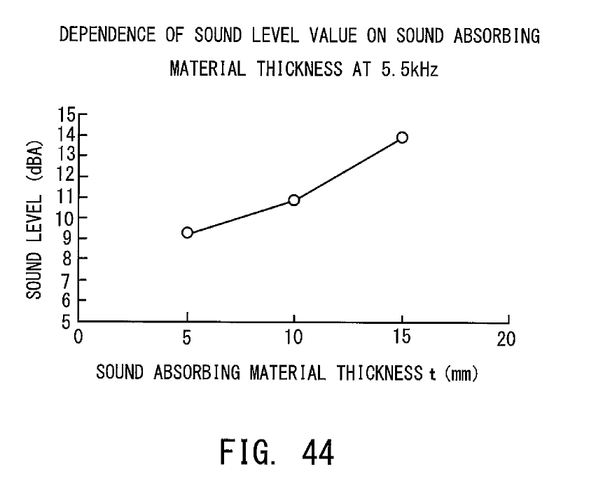

FIGS. 42 to 44 are views illustrating noise levels of 1 kHz, 3.5 kHz and 5.5 kHz which noise levels are read from the FIG. 41, respectively.

As found in these figures, the thinner the thickness of the sound absorbing member is, the larger the effects in which the noise levels of the higher frequencies are reduced become.

Accordingly, when a discharge silencer in which a sound absorbing member made of foamed material is applied is used, by adjusting the thickness or the length thereof, it is possible to effectively reduce noises of a targeted frequency band.

In other words, by applying a fan of the air dynamic bearing, it is possible to achieve significant downsizing and weight reduction, and with respect to noises which become a problem when such a fan of the air dynamic bearing is applied, it is possible to effectively reduce the noises by applying a discharge silencer. In other words, by a combination of a fan of the air dynamic bearing and a discharge silencer, it is possible to achieve the compatibility between downsizing, weight reduction and noise reduction in a high order.

This ends the explanations of the CPAP apparatus 1H according to the eighth embodiment, and in the following, embodiments of a ninth embodiment and embodiments thereafter will be explained. Incidentally, in the drawings illustrating each of the ninth embodiment and the embodiments after the ninth embodiment, for the convenience of understanding, components and the like functionally corresponding to those included in the CPAP apparatus according to the eighth embodiment even though there are differences in shapes and the like are illustrated while being assigned with signs same as those put in each of the drawings used for the explanations of the eight embodiment, and configuration portions distinctive to each embodiment will be explained.

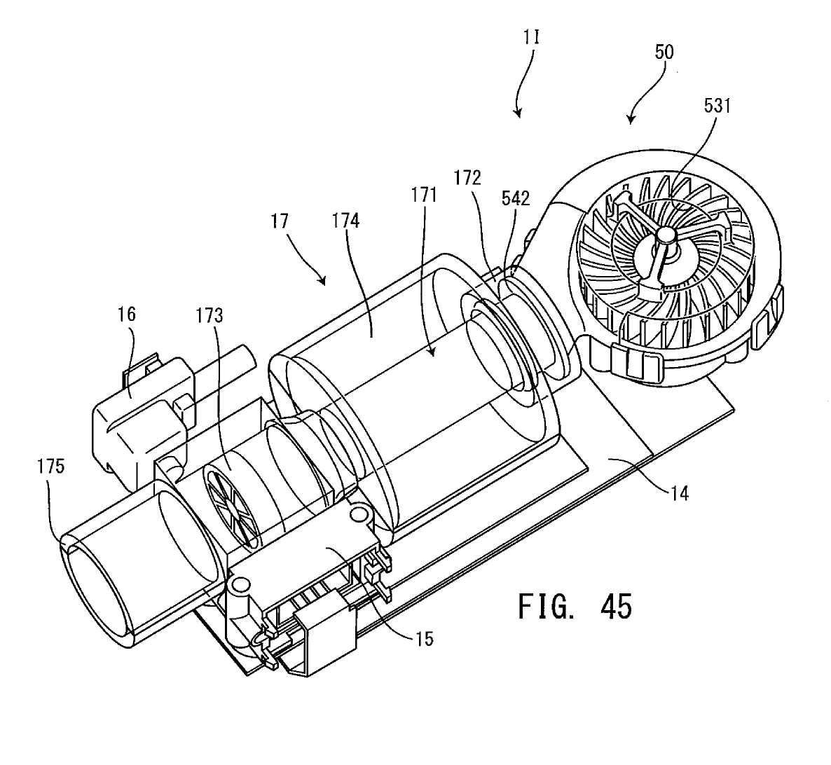

FIG. 45 is a transparent view when the case and the suction silencer are removed from the CPAP apparatus according to the ninth embodiment, and a fan, a discharge silencer and the like are viewed obliquely from above. This FIG. 45 is a figure corresponding to FIG. 30 which is used for the explaining the CPAP apparatus according to the eight embodiment.

A discharge silencer 17 included in a CPAP apparatus 1I according to the ninth embodiment includes a sound absorbing member 174 on a side of the turbofan 50, and a rectifying element 173 is arranged on a side more downstream in an air flow than the sound absorbing member 174. A flow sensor 15 and a pressure sensor 16 are coupled to a downstream side of the rectifying element 173.

As illustrated in here, each one of the sound absorbing member 174 and the rectifying element 173 may arranged in an upstream side or a downstream side to the other.

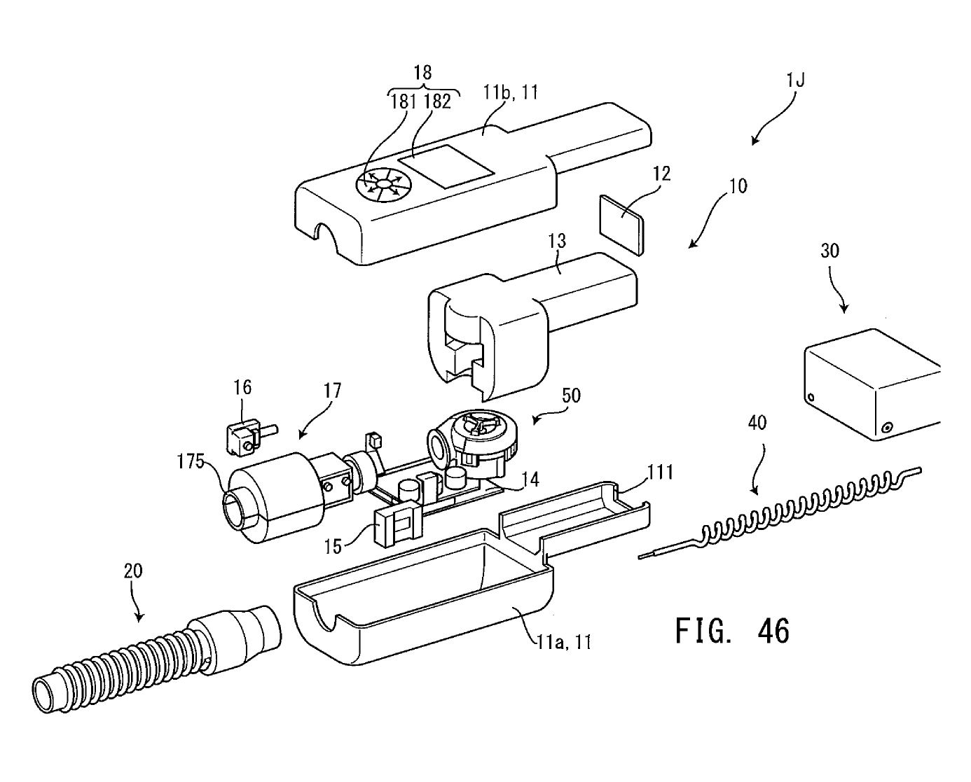

FIG. 46 is an exploded perspective view of a CPAP apparatus according to a tenth embodiment.

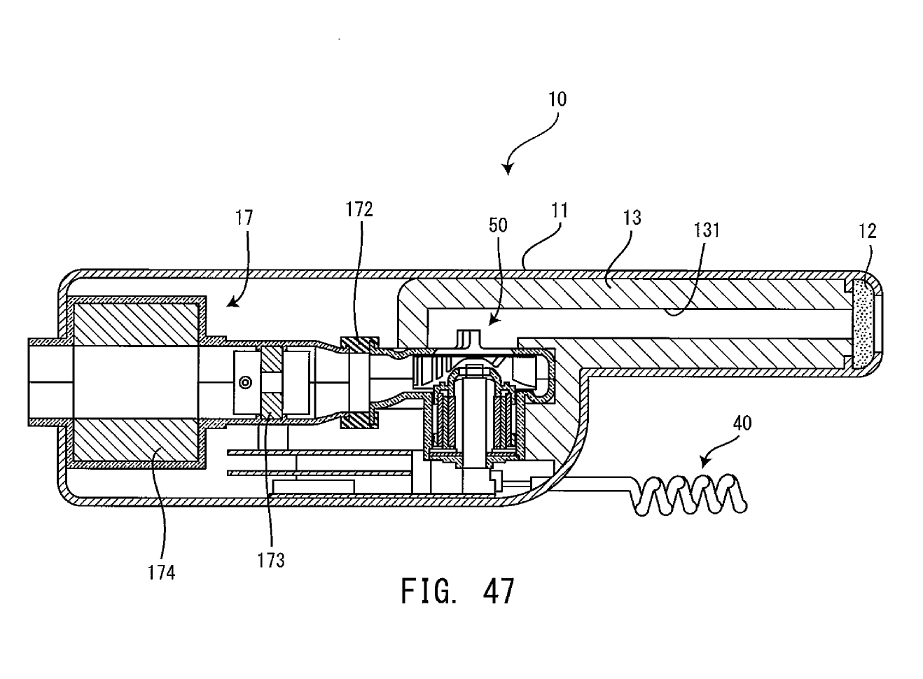

In addition, FIG. 47 is a sectional view of an air blowing unit of the CPAP apparatus whose exploded perspective view is illustrated in FIG. 46.

An air blowing unit of a CPAP apparatus 1J according to the tenth embodiment illustrated in FIG. 46 and FIG. 47 includes a case 11 which is formed to have a squarish shape compared to the air blowing unit (see FIG. 27) of the CPAP apparatus 1H according to the above-described eighth embodiment. In the case of the air blowing unit according to the eighth embodiment, the air blowing unit has the case having a round shape so as to roll according to posture changing of a patient, and however, in here, for example, it is assumed that the air blowing unit is placed on a kakebuton (a bed cover) and the like of a patient while being in bed, and posture stability of the air blowing unit 10 is considered to be important. When a patient changes its posture by turning over and the like, the air blowing unit 10 according to the tenth embodiment follows the posture changing of the patient mainly by moving slidably.

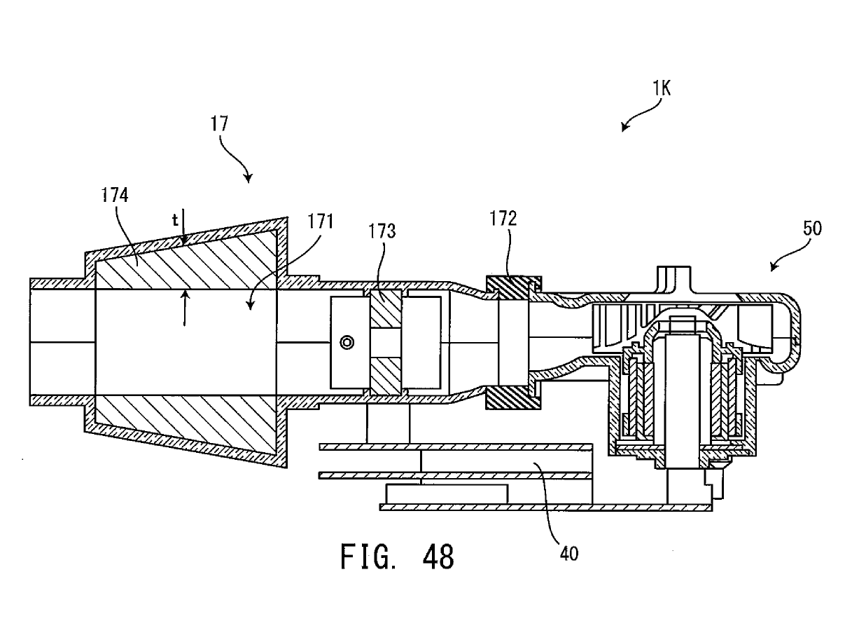

FIG. 48 is a sectional view of a fan and a discharge silencer of a CPAP apparatus according to a eleventh embodiment.

In a case of the CPAP apparatus 1K according to this eleventh embodiment, a sound absorbing member 174 included in a discharge silencer 17 in an air blowing unit 10 is formed such that the thickness thereof becomes continuously thinner from an upstream side toward a downstream side in an air flow. As easily conjectured from the above-described experimental data, specifically, the experimental data when the thickness t of the sound absorbing member is changed which data is illustrated in FIGS. 41 to 44, by changing the thickness t, it is expected that noises in a broad frequency band are reduced.

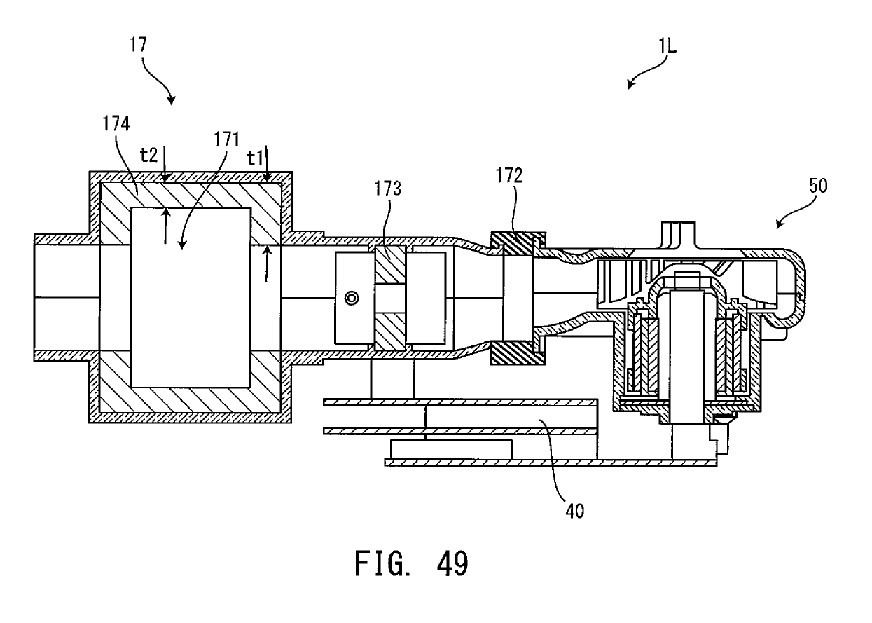

FIG. 49 is a sectional view of a fan and a discharge silencer of a CPAP apparatus according to a twelfth embodiment.

In a case of a CPAP apparatus 1L according to this twelfth embodiment, a sound absorbing member 174 of a discharge silencer 17 in an air blowing unit 10 has the thickness t which is thick at both ends (t=t1) and thin at a center (t=t2). With this, similarly to the case of the eleventh embodiment illustrated in FIG. 48, it is expected that noises in a broad frequency band are reduced. In addition, in the case of the discharge silencer 17 according to this twelfth embodiment, the sectional area of a discharge path 171 changes, and also with this, a noise reduction effect is expected.

Incidentally, the examples including the air dynamic bearing have been explained in here, and however, one including an oil dynamic bearing also may achieve similar effects.

In addition, each of the above-described embodiments is a CPAP apparatus which is supposed to be used while being combined with a mask. The CPAP apparatus according to the present invention may be also applied to a CPAP apparatus of a type which is used while being combined with a nasal cannula instead of a mask.

### REFERENCE SIGNS LIST

1A-1L CPAP apparatus
10 Air blowing unit
11 Case
11a Case lower section
11b Case upper section
12 Air filter
13 Suction silencer
14 Control board
15 Flow sensor
16 Pressure sensor
17 Discharge path
18 User interface
19 Surface filter member
20 Hose
30 Battery case
40 Cable
50 Turbofan
51 Stator
52 Rotator
53 Upper cover
60 Remote controller
61 User interface
62 Control section
63 Setting signal generating section
64 Communication module
71 External shell case
80 Movable joint
97 Discharge silencer
111 Air suction port
112 Air discharge port
113 Projection
115 Boat-form bottom surface
116 Upper surface
131 Air flow path
141 MPU
142 Motor drive circuit
143 Communication module
181 Operation button
182 Display screen
200 Mask
201 Leaking apertures
300 Patient
301 Battery
302 Connecting terminal
310 Patient head
511 Shaft base
512 Thrust magnet (inside)
513 Circuit board
514 Coil base
516 Chest
516a Leg
517 Coil
518 Case
519 Screw
521 Hub
522 Thrust magnet (outside)
524 Sleeve
526 Magnet
527 Back yoke
528 Balance ring
529 Blade
529a Bypass opening
531 Air receiving port
532 Stopper
541 Sintered body
542 Air blowing port
543 Locking projection
600 Anechoic room
601 Fan
602 Flowmeter
603 Pressure gauge
604 Hose
605 Dummy head
606 Respiration simulator
607 Noise meter
611 Operation button
612 Display screen
711 External case rib
712 External case stay
713 External shell case outer surface
971 Discharge path
972 Joint
973 Rectifying element
974 Sound absorbing member
975 Air discharge port

1. A CPAP apparatus comprising:
an air blowing unit that includes a housing which has an air suction port, and that includes a fan which has an air receiving port and an air blowing port, is provided with a fluid dynamic bearing, and receives air from the receiving port which air is suctioned from the suction port into the housing to send out the air from the air blowing port; and
a hose that connects the air blowing unit with an air intake port of a nasal cannula or a mask which is attached to the head of a patient so as to cover an external naris or a nose of the patient and supplies the air taken in from the air intake port to a respiratory tract of the patient, and that sends the air sent out from the air blowing unit from the air intake port to the nasal cannula or the mask, wherein
the air blowing unit is supported at a position away from the head of the patient by other than the hose, and the hose is a hose having such a length that exerts a force to the air blowing unit via the hose when the patient changes a posture thereof while lying, and the air blowing unit receives the force to change a position or a posture thereof.

2. The CPAP apparatus according to claim 1, wherein
the air blowing unit further includes a control circuit which receives an instruction by wireless communication and controls the fan based on the instruction, and
the CPAP apparatus further comprises a remote controller that gives an instruction to the control circuit by wireless communication.

3. The CPAP apparatus further comprising a movable joint between the air blowing unit and the hose.

4. The CPAP apparatus according claim 1, wherein the housing includes a plurality of air suction ports.

5. The CPAP apparatus according to claim 1, wherein the housing includes a guard section which prevents the air suction port from being blocked.

6. The CPAP apparatus according to claim 1, wherein the air blowing unit further includes a discharge silencer which reduces noise as the air flows which air is sent out from the air blowing port by the fan coupled to the air blowing port flows.

7. The CPAP apparatus according to claim 6, wherein the discharge silencer includes a sound absorbing member made of foamed material.

8. The CPAP apparatus according to claim 1, wherein the air blowing unit further includes a suction silencer which includes a sound absorbing member in which a suction path to guide the air suctioned into the housing from the air suction port to the air receiving port, and supports the fan such that the suction silencer contains the fan.

9. The CPAP apparatus according to claim 1, wherein the air blowing port and the discharge silencer are connected with each other by a joint formed by an elastic body.
